(19) United States
(12) Patent Application Publication
(10) Pub. No.: US 2010/0113643 A1
(43) Pub. Date: May 6, 2010

(54) CURATIVES FOR EPOXY ADHESIVE COMPOSITIONS

(75) Inventor: Stephen M. Dershem, San Diego, CA (US)

Correspondence Address: The Law Office of Jane K. BABIN,;Professional Corporation, c/o Intellevate, P.O. Box 52050, Minneapolis, MN, 55402, US

(73) Assignee: DESIGNER MOLECULES, INC., San Diego, CA (US)

(21) Appl. No.: 12/595,505

(22) PCT Filed: Apr. 9, 2008
(86) PCT No.: PCT/US08/59804

§ 371 Date: Oct. 9, 2009

Related U.S. Application Data
(60) Provisional application No. 60/922,412, filed on Apr. 9, 2007.
(60) Provisional application No. 60/930,166, filed on May 15, 2007.

(51) Int. Cl. C09J 4/00; C07D 205/04; C07D 209/48; C07C 69/00; C07C 69/76; C07F 7/10; C08G 59/14; C08L 63/02; A61K 6/087; A61K 8/72

(52) U.S. Cl. 523/120; 548/520; 548/462; 548/542; 560/139; 560/103; 556/419; 525/533; 523/455; 523/105; 523/177

(57) ABSTRACT

The invention provides epoxy and oxetane compositions including the novel acyloxy and N-acyl curing agents described herein. Use of invention curing agents result in cured adhesive compositions with remarkably increased adhesion and reduced hydrophilicity when compared to resins cured with other types of curing agents. Furthermore, the curatives of this invention do not interfere with free-radical cure and are thus suited for use in hybrid cure thermoset compositions.

### RELATED APPLICATIONS

This application claims the benefit of priority under 35. U.S.C. §119 of U.S. Provisional Application Ser. No. 60/922,412, filed Apr. 9, 2007 and to U.S. Provisional Application Ser. No. 60/930,166, filed May 15, 2007, the disclosures of which are incorporated herein by reference in their entirety.

### FIELD OF THE INVENTION

The present invention relates to thermosetting compositions, methods of preparation and uses therefor. In particular, the present invention relates to thermosetting compounds and compositions containing epoxy and oxetane resins, and acetoxy, acyloxy, and N-acyl curatives therefore.

### BACKGROUND OF THE INVENTION

The properties of cured epoxy resins are often influenced dramatically by the curing agent that is added to the formulation. Accordingly, much research effort has been directed towards developing curing agents that can enhance the properties of the cured resin. Phenols, anhydrides, thiols and amines have generally been used as curing agents in epoxy resins. While useful, these curing agents are not without certain drawbacks. Thus, a continuing need exists for new epoxy curing agents.

### SUMMARY OF THE INVENTION

The compounds described herein provide a significant advance in the field of epoxy chemistry. The present invention provides curative compounds that impart outstanding properties for epoxy and oxetane cure. More specifically, the invention provides acetoxy, acyloxy, and N-acyl curatives, as well as epoxy and oxetane resin compositions that include these curatives. Significantly, the resulting thermosets can have such desirable properties as reduced hydrophilicity, decreased viscosity, increased thermal resistance, and increased hydrolytic stability. In contrast to phenolic curatives, the acetoxy, acyloxy, and N-acyl curatives described herein do not interfere with free-radical cure chemistry. This feature dramatically expands opportunities for hybrid cures (i.e. those that combine ring opening addition cures of epoxies and/or oxetanes with any of the free-radically curable monomers).

When an N-acyl compound of the invention is used as a curative, an N-acylated imide co-cure with an epoxy resins result in a polyimide. Polyimides are considered to be one of the highest performance resins with respect to thermal resistance. Certain compounds of this invention, therefore, provide a means of converting epoxy monomers into polyimide resins.

One feature of the N-Acyl curatives described herein that makes them especially valuable is their high level of reactivity. They can, for example, be used to cure aliphatic and cycloaliphatic epoxies Anhydrides have previously been the only class of curatives available for the aliphatic and cycloaliphatic epoxies. The N-acyl compounds of this invention can be used to provide thermosets with superior hydrolytic and thermal resistance compared to adhesives, coatings, encapsulants, or matrix resins that utilize anhydride curatives. The N-acyl curatives of this invention have a further advantage in that, unlike anhydrides, they do not react with moisture at room temperature. This can be an important consideration for shelf-life and product performance in humid environments.

The compounds of the invention are useful for single lay-up, two stage cures. In certain of these embodiments, a di-functional epoxy or oxetane monomer may be cured with a di-functional acyloxy compound to form a thermoplastic intermediate. The initially formed polymer may then be cross-linked to a final thermoset in a second step. This chemistry is, therefore, the ideal platform for b-stageable adhesives.

The compounds of the invention are also useful in a variety of other applications. Invention compounds can be used in automotive, marine, and aerospace coatings and adhesives. The properties of certain invention compounds make these compounds suitable for use in dental matrix resins and adhesives. Invention compounds can also be used as components of matrix resins and composites used in sports equipment, automotive bodies, and boat construction, such as those incorporating carbon fiber and/or fiberglass reinforcements. The compounds of the present invention also have attractive properties for use in adhesives for diverse industrial applications, such as thread-lock materials and building materials.

In general, epoxies are known for their excellent adhesion, chemical and heat resistance, good to excellent mechanical properties and very good electrical insulating properties, but many of these properties can be modified. For example, although epoxies are typically electrically insulating, epoxies filled with silver or other metals can be electrically conductive.

The curatives of this invention can be used, for example, with aliphatic, cycloaliphatic, glycidyl ether, glycidyl ester, and glycidyl amine epoxies, as well as with combinations thereof. Furthermore, these compounds may be used as curatives for oxetane monomers.

Accordingly, the present invention provides curatives for epoxy or oxetane resins having the structure of:

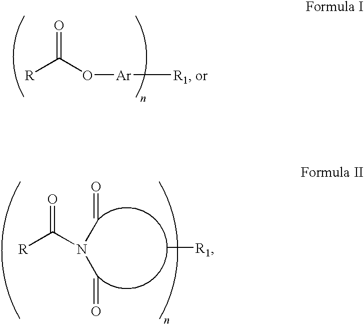

where R and R₁ are each independently substituted or unsubstituted aliphatic, heteroaliphatic, aromatic, heteroaromatic, siloxane, maleimido, cinnamyl; Ar is substituted or unsubstituted aryl or heteroaryl having from 6 to about 20 carbon atoms; and n is 1 to about 11. In certain embodiments, R and R₁ are each independently substituted or unsubstituted alkyl, cycloalkyl, alkenyl, aryl, or heterocyclic. In other embodiments, Ar is substituted or unsubstituted C₆ to about C₁₁ aryl or heteroaryl. In certain aspects of the invention, n is 1 to about 6.

In certain embodiments, the curatives of the present invention are liquids at room temperature.

Curatives provided by the present invention include, but are not limited to:

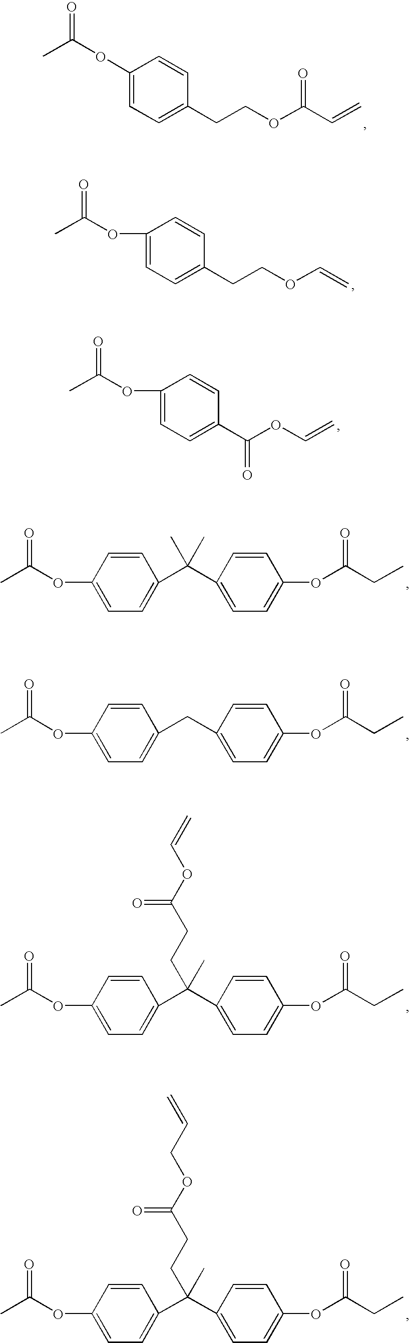

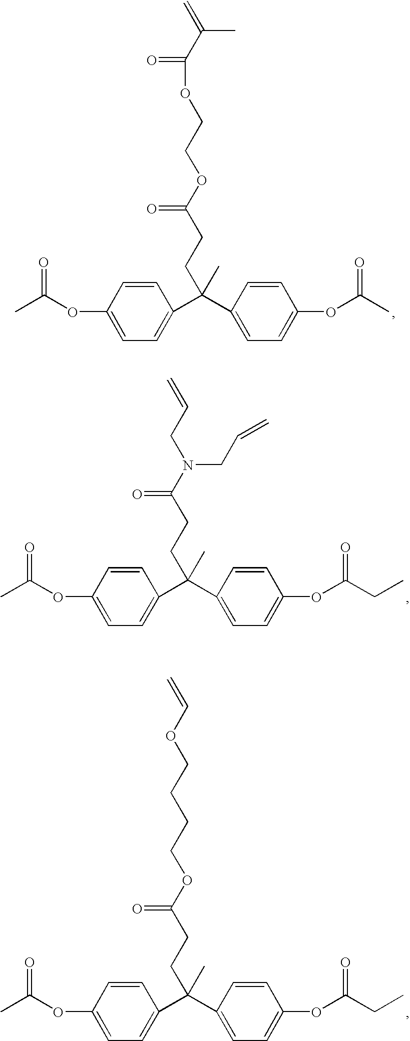

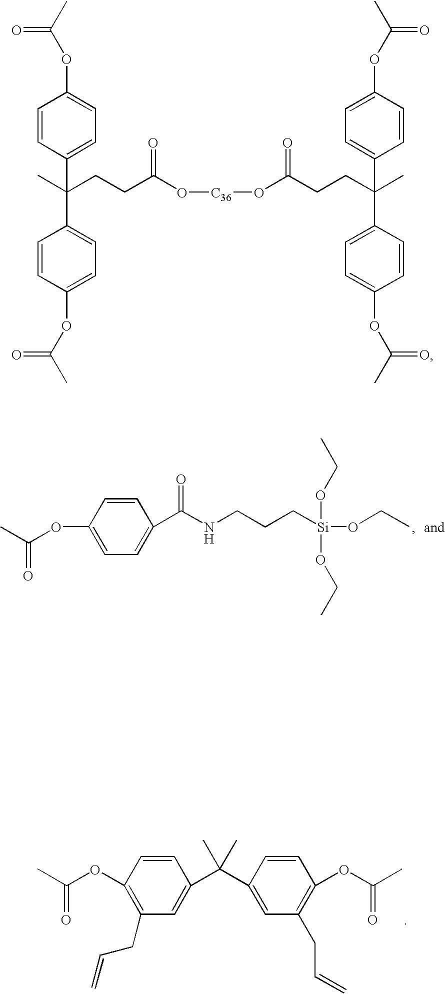

Curatives provided by the invention also include:

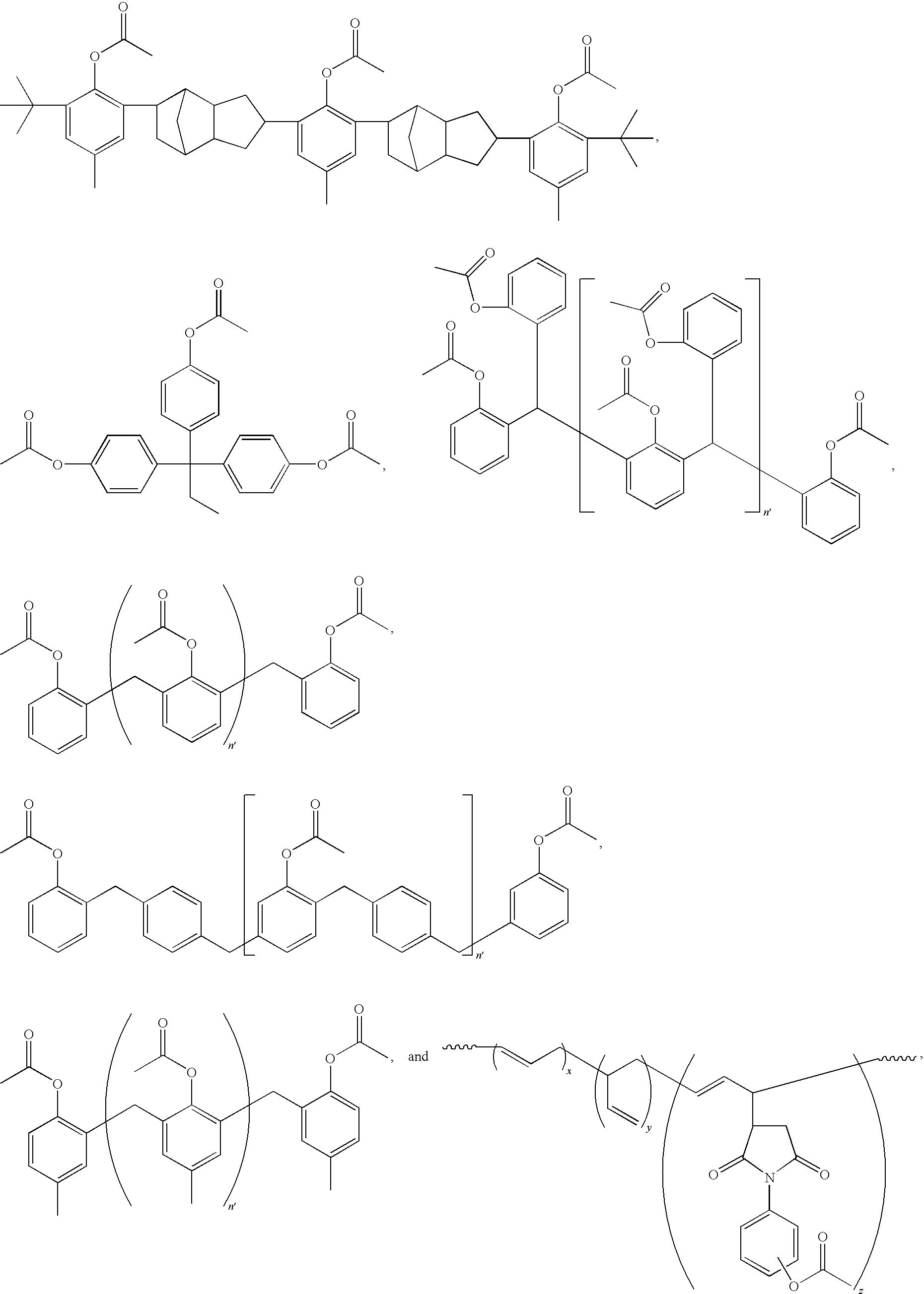

where n′ is 0 to 10; x is 4 to about 50; y is 4 to about 50; and z is 2 to about 40.

The present invention also provides poly-N-acyl curatives including:

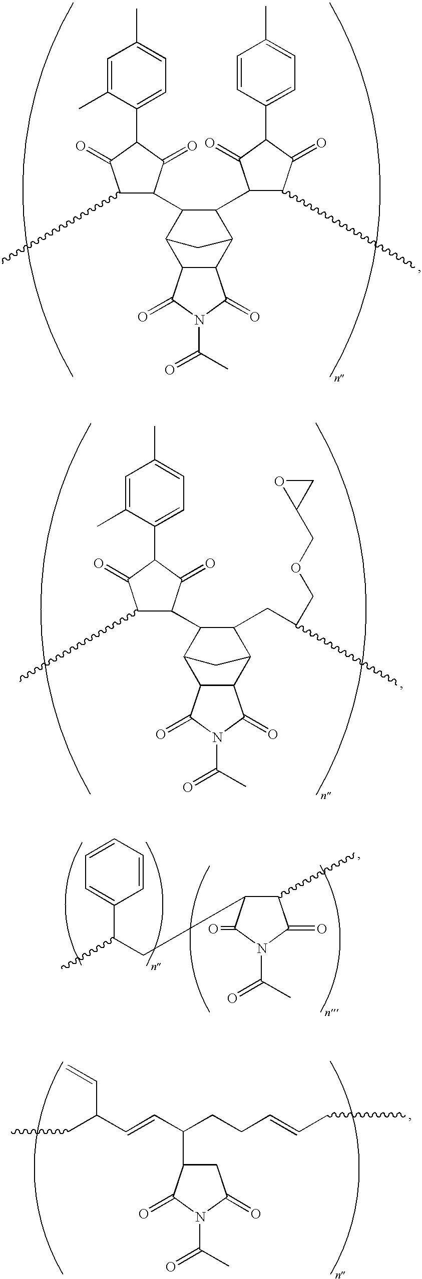

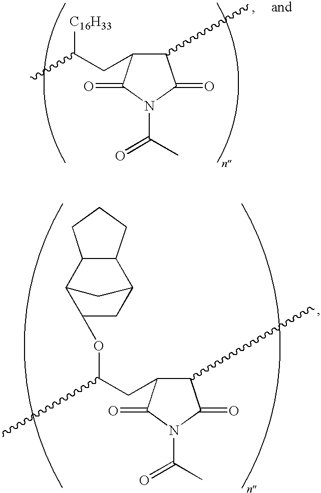

where each n″ and n′″ is independently 1 to about 10.

Curatives according to the invention also include, for example:

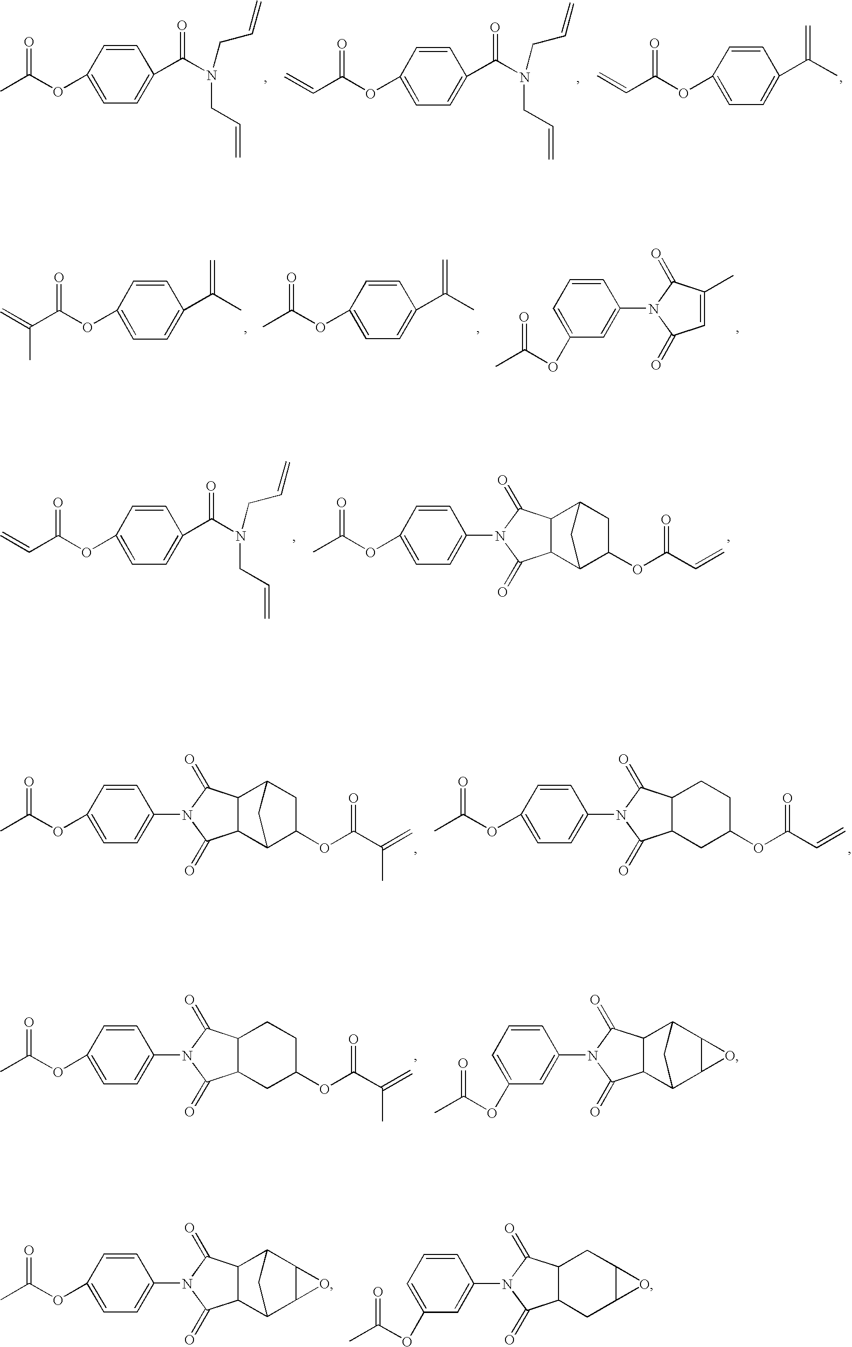

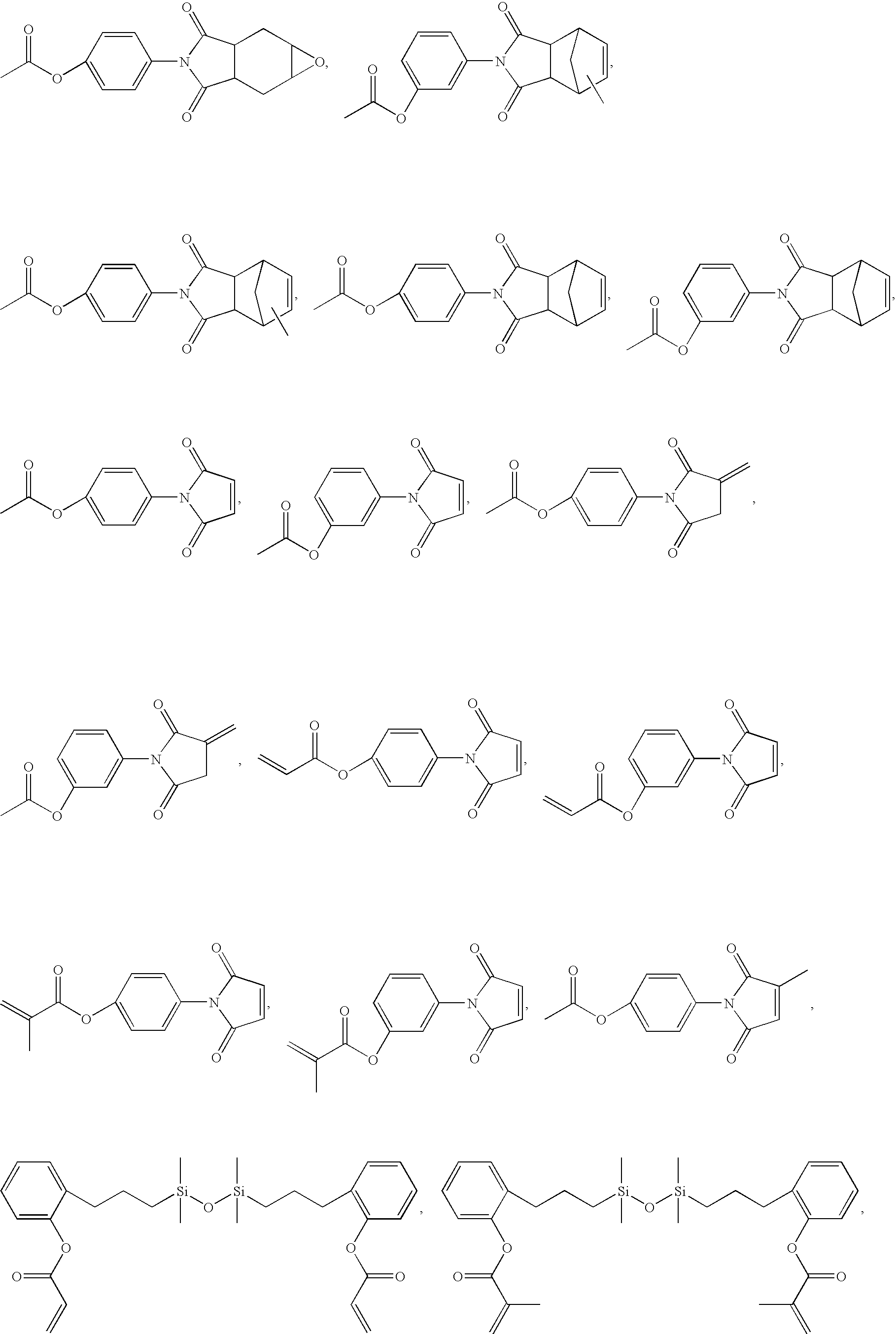

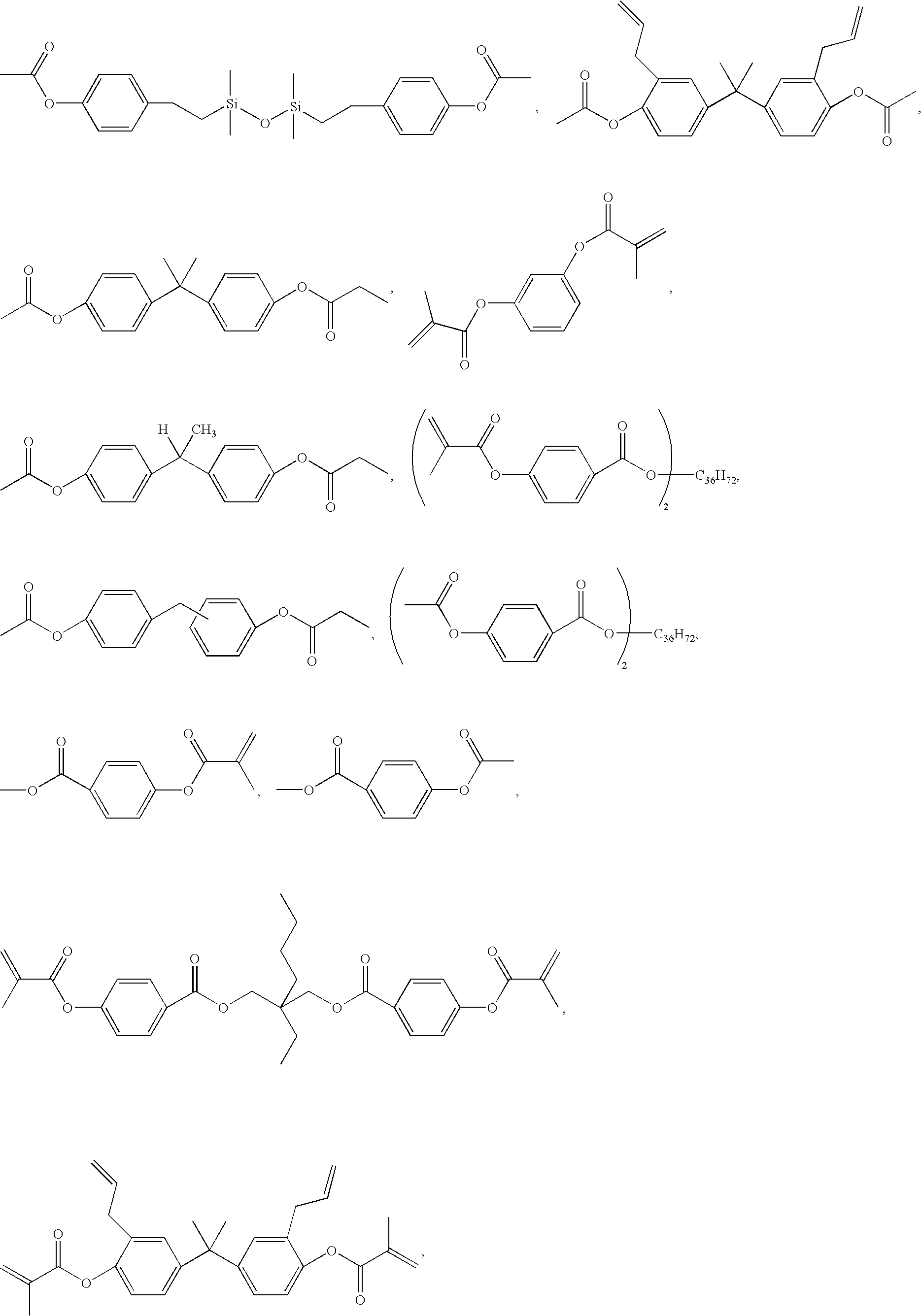

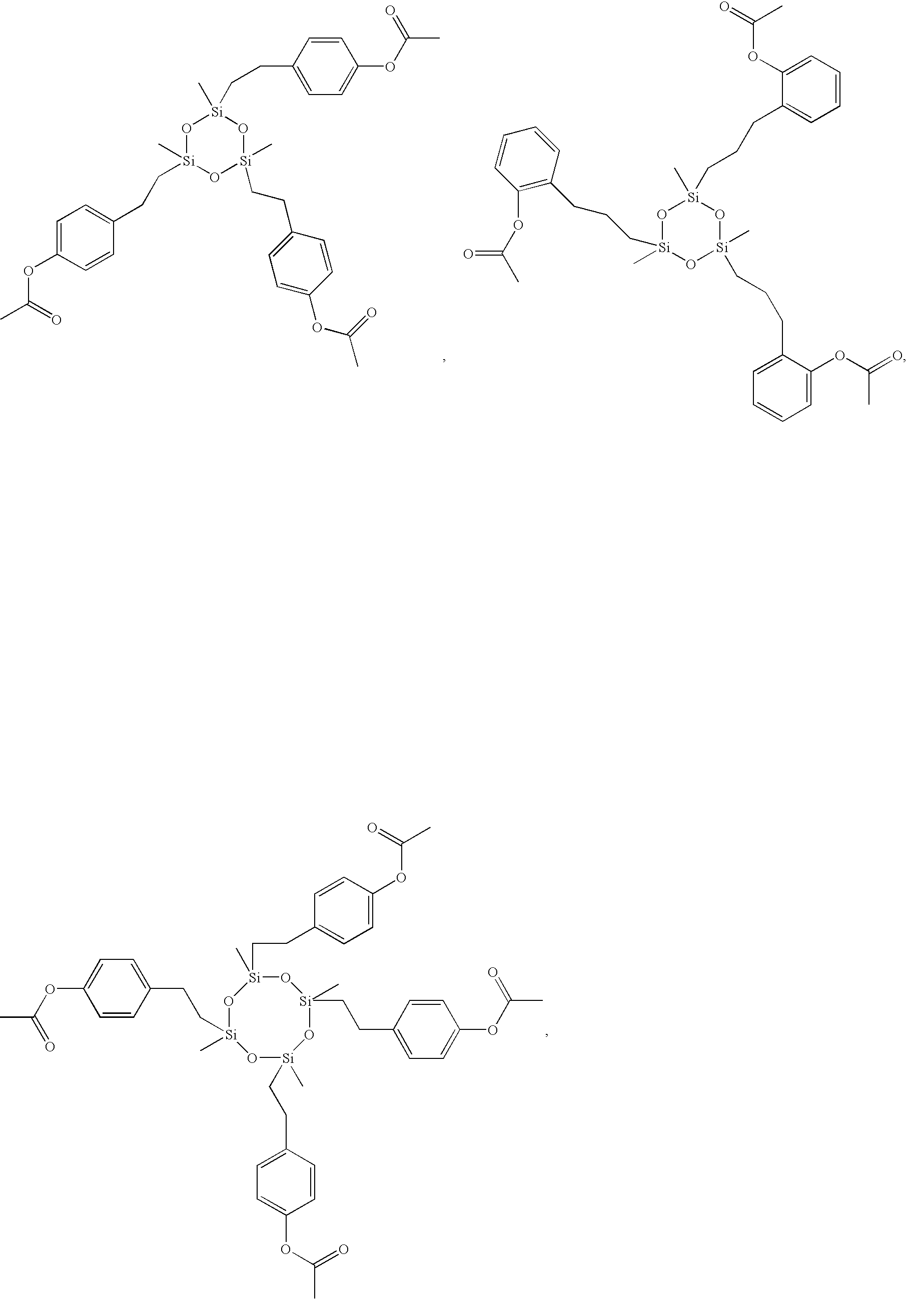

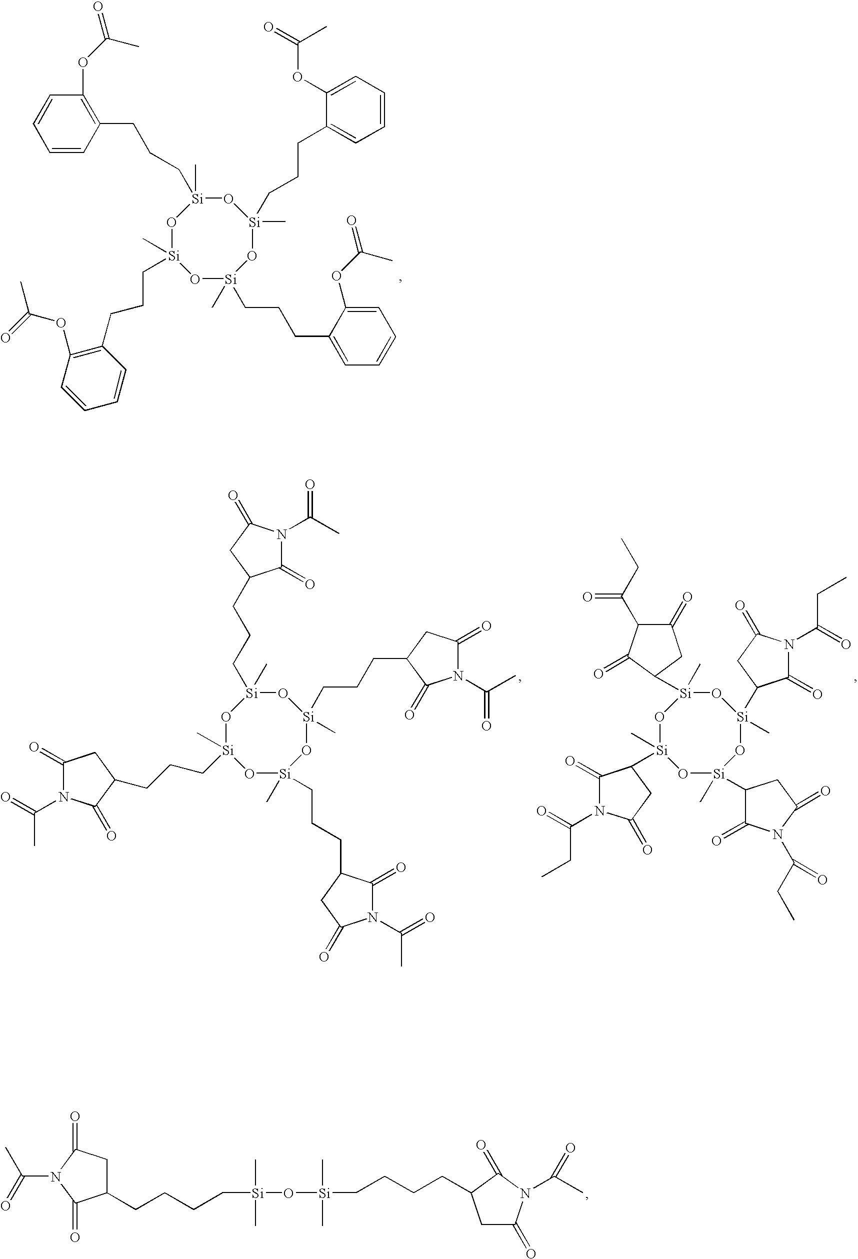

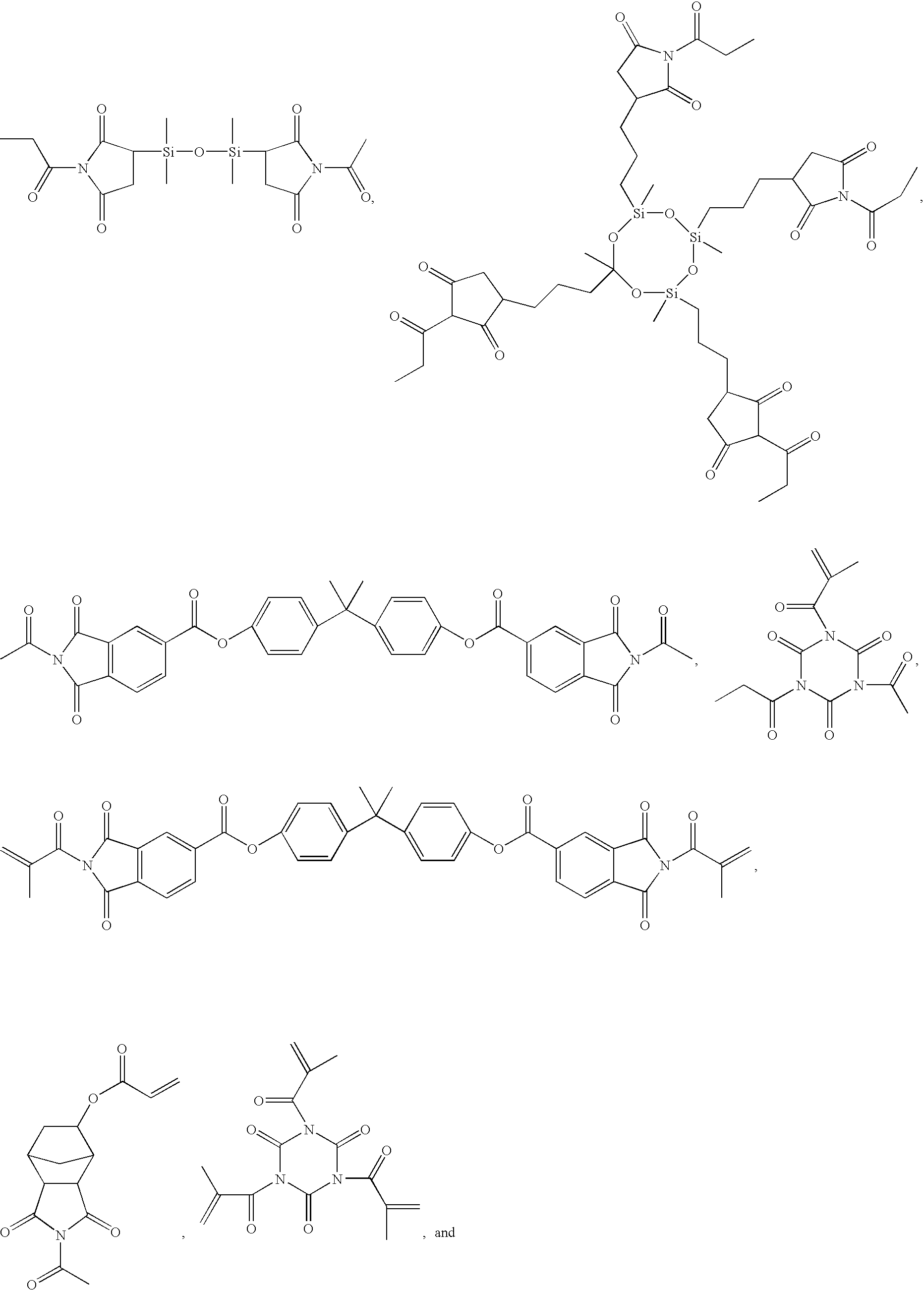

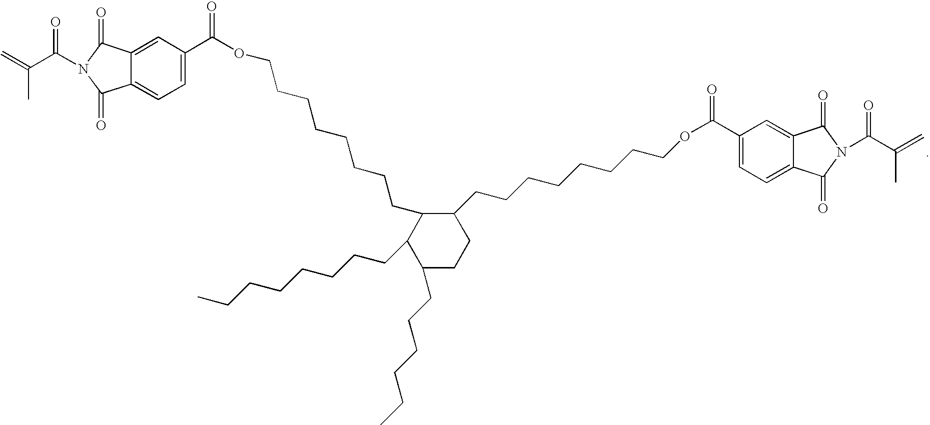

The present invention also provides compositions that include an epoxy or oxetane resin and one or more of the curatives described above. In certain embodiments, the epoxy includes at least one of a glycidyl ether epoxy, a cycloaliphatic epoxy, and an aliphatic epoxy. The glycidyl ether epoxy can be, for example, a glycidyl ether of a phenol, an amine, an alcohol, or an isocyanurate; a trisglycidyl ether of a phenolic compound; a glycidyl ether of a cresol formaldehyde condensate; a glycidyl ether of a phenol formaldehyde condensate; a glycidyl ether of a cresol dicyclopentadiene addition compound; a glycidyl ether of a phenol dicyclopentadiene addition compound; a glycidyl ether of a fused ring polyaromatic phenol; a diglycidyl ether; a glycidyl ether of an aliphatic alcohol; a glycidyl ether of a polyglycol; a glycidyl derivative of an aromatic amine; an ester linked epoxy; a phenyl glycidyl ether; a cresol glycidyl ether; a nonylphenyl glycidyl ether; a p-tert-butylphenyl glycidyl ether; a diglycidyl ether or a trisglycidyl ether of bisphenol A, bisphenol F, ethylidinebisphenol, dihydroxydiphenyl ether, N,N′-disalicylal-ethylenediamine, triglycidyl-p-aminophenol, N,N,N′,N′-tetraglycidyl-4,4′-diphenylmethane, triglycidyl isocyanurate, bis(4-hydroxyphenyl)sulfone, bis(hydroxyphenyl)sulfide, 1,1-bis(hydroxyphenyl)cyclohexane, 9,19-bis(4-hydroxyphenyl)fluorene, 1,1,1-tris(hydroxyphenyl)ethane, tetrakis(4-hydroxyphenyl)ethane, trihydroxytritylmethane, 4,4′-(1-alpha-methylbenzylidene)bisphenol, 4,4′-(1,3-componentthylethylene)diphenol, componentthylstilbesterol, 4,4′-dihyroxybenzophenone, resorcinol, catechol, or tetrahydroxydiphenyl sulfide; a glycidyl ether of a dihydroxy naphthalene, 2,2′-dihydroxy-6,6′-dinaphthyl disulfide, or 1,8,9-trihydroxyanthracene; a diglycidyl ether of 1,4 butanediol; a diglycidyl ether of diethylene glycol; a diglycidyl ether of neopentyl glycol; a diglycidyl ether of cyclohexane dimethanol; a diglycidyl ether of tricyclodecane dimethanol; a trimethyolethane triglycidyl ether; a glycidyl ether; a trimethyol propane triglycidyl ether; a glycidyl ether of Heloxy 84™; a glycidyl ether of Heloxy 32™; a polyglycidyl ether of castor oil; polyoxypropylene diglycidyl ether; Heloxy 71; and/or glycidyl methacrylate.

In certain embodiments, the cycloaliphatic epoxy ether can include a cyclohexene oxide; a 3-vinylcyclohexene oxide; a vinylcyclohexene dioxide; a dicylcopentadiene dioxide; a tricyclopentadiene dioxide; a tetracyclopentadiene dioxide; a norbornadiene dioxide; a bis(2,3-epoxycyclopentyl)ether; a limonene dioxide; 3′,4′-epoxycyclohexamethyl-3,4-epoxycyclohexanecarboxylate; a 3,4-epoxycyclohexyloxirane; a 2(3′,4′-epoxycyclohexyl)-5,1″-spiro-3″,4″-epoxycyclohexane-1,3-dioxane; and/or a bis(3,4-epoxycyclohexamethyl)adipate.

In other embodiments, the aliphatic epoxy can include an epoxidized polybutadiene; an epoxidized polyisoprene; an epoxidized poly(1,3-butadiene-acrylonitrile); an epoxized soybean oil; an epoxidized castor oil; a dimethylpentane dioxide; a divinylbenzene dioxide; a butadiene dioxide; and/or a 1,7-octadiene dioxide.

The compositions of the invention include compositions useful as adhesives, coatings, matrix resins and composite resins. In certain embodiments, the composition is a die paste adhesive that includes a filler. In other embodiments, the composition is an industrial or marine coating that includes a filler, an extender and/or a pigment.

Also contemplated by the invention are compositions including industrial, marine, automotive, airline, aerospace, sporting goods, medical and dental matrix resins. In yet other aspects of the invention, the compositions can be composite resins that include for example, carbon fiber, fiberglass and/or silica.

Certain compositions of the invention, such as adhesives, can also include additional compounds such as acrylates, methacrylates, maleimides, vinyl ethers, vinyl esters, styrenic compounds, allyl functional compounds, phenols, anhydrides, benzoxazines, and oxazolines.

The present invention also provides assemblies that include a first article adhered to a second article by a cured aliquot of the adhesive composition described above. Also provided are articles of manufacture coated with a cured layer of one of the compositions described above, such as a watercraft, automobile or airplane parts. In other embodiments of the invention, articles of manufactures can be comprised of a cured amount of a composition described herein, such as an industrial, marine, automotive, airline, aerospace, sporting goods, medical or dental article. Such articles of manufacture can also include fillers, extenders, pigments and/or reinforcing materials along with the compositions disclosed herein.

Method for attaching a first article to a second article are also provided by the invention, including the steps of applying an adhesive composition as disclosed above to the first article, the second article, or both the first article and the second article; then contacting the first article and second article, such that the first article and the second article are separated only by the adhesive composition, which results in the formation of an assembly. Upon curing of the adhesive composition, the first article is adhesively attached to the second. In certain embodiments, the adhesive composition includes a free-radical curable monomer and curing is by a hybrid thermosetting and free-radical cure.

The present invention also provides methods for adhesively attaching a semiconductor die to a substrate including the steps of applying the adhesive composition of the invention to the substrate, the semiconductor die, or the substrate and the semiconductor die; contacting the substrate and the die, such that the substrate and the die are separated only by the adhesive composition, to form an assembly; and then curing the adhesive composition, which results in adhesively attaching the semiconductor die to the substrate. In certain embodiments, the adhesive composition includes a free-radical curable monomer and curing is by a hybrid thermosetting and free-radical cure.

The present invention also contemplates use of the acyl curatives described above in methods for increasing the adhesion, decreasing the viscosity, reducing weight loss, and decreasing the hydrophilicity of an epoxy or oxetane resin, by combining an acyl curative of invention with the epoxy or oxetane resin.

### BRIEF DESCRIPTION OF THE DRAWINGS

FIG. 1 shows a scheme for the first step of a b-staging procedure represented by a chain extension and termination sequence.

FIG. 2 shows a scheme for the final step in a b-staging procedure, which involves a thermal cure to cross-link b-staged functional oligomers.

### DETAILED DESCRIPTION OF THE INVENTION

Unless specific definitions are provided, the nomenclatures utilized in connection with, and the laboratory procedures and techniques of analytical chemistry, synthetic organic and inorganic chemistry described herein are those known in the art. Standard chemical symbols are used interchangeably with the full names represented by such symbols. Thus, for example, the terms “hydrogen” and “H” are understood to have identical meaning Standard techniques may be used for chemical syntheses, chemical analyses, and formulation. The foregoing techniques and procedures can be generally performed according to conventional methods well known in the art.

It is to be understood that both the foregoing general description and the following detailed description are exemplary and explanatory only and are not restrictive of the invention claimed. As used herein, the use of the singular includes the plural unless specifically stated otherwise. As used herein, “or” means “and/or” unless stated otherwise. Furthermore, use of the term “including” as well as other forms, such as “includes,” and “included,” is not limiting.

The section headings used herein are for organizational purposes only and are not to be construed as limiting the subject matter described.

The invention provides novel acetoxy, acyloxy, and N-acyl curing agents useful in a variety of epoxy adhesive formulations. As used herein, “acyloxy” refers compounds having at least one moiety of the following general structure:

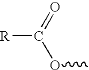

“Acetoxy”, according to the present invention, refers to compounds having at least one moiety of the following general structure:

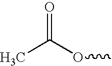

“N-acyl”, according to the present invention, refers to compounds having at least one moiety of the following general structure:

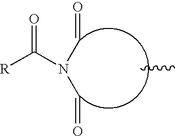

According to one embodiment of the invention, epoxy curing agents having the structure of Formulae I, and II, below, are provided:

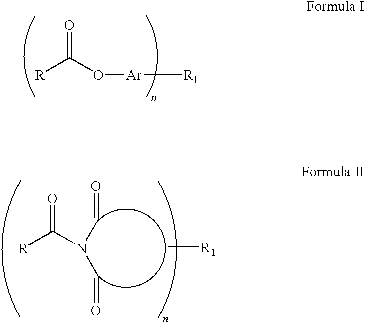

where R and R₁ are each independently substituted or unsubstituted aliphatic, heteroaliphatic, aromatic, heteroaromatic, siloxane, maleimido, or cinnamyl; Ar is substituted or unsubstituted aryl or heteroaryl having from 6 to about 20 carbon atoms; and n is 1 to about 11.

“About” as used herein means that a number referred to as “about” comprises the recited number plus or minus 1-10% of that recited number. For example, “about” 100 degrees can mean 95-105 degrees or as few as 99-101 degrees depending on the situation. Whenever it appears herein, a numerical range such as “1 to 20” refers to each integer in the given range; e.g., “1 to 20 carbon atoms” means that an alkyl group can contain only 1 carbon atom, 2 carbon atoms, 3 carbon atoms, etc., up to and including 20 carbon atoms, although the term “alkyl” also includes instances where no numerical range of carbon atoms is designated).

In certain embodiments, R and R₁ are each independently substituted or unsubstituted alkyl, cycloalkyl, hetero alkyl, alkenyl, heteroalkenyl, aryl, heterocyclic siloxane, maleimido, or cinnamyl.

In certain aspects of the invention, R and R₁ are each independently substituted or unsubstituted alkyl, cycloalkyl, alkenyl, aryl, or heterocyclic. According to some embodiments, at least one of R and R₁ is a C₁ to about C₂₀ substituted or unsubstituted alkyl, cycloalkyl, alkenyl, or aryl.

In other aspects, R and R₁ are each independently substituted or unsubstituted siloxane or maleimide.

In yet other embodiments, Ar is substituted or unsubstituted C₆ to about C₁₁ aryl or heteroaryl. In still further embodiments, Ar is phenyl, benzyl, tolyl, or xylyl.

According to the present invention, n is 1 to about 11. In some embodiments, n is about 2 to about 10. In yet other embodiments, n is about 4 to about 8. While in still further embodiments, n is 1 to about 6.

As used herein, “alkyl” refers to straight or branched chain hydrocarbyl groups having from 1 up to about 500 carbon atoms.

“Substituted alkyl” refers to alkyl moieties bearing substituents including, but not limited to, alkyl, alkenyl, alkynyl, hydroxy, oxo, alkoxy, mercapto, cycloalkyl, substituted cycloalkyl, heterocyclic, substituted heterocyclic, aryl, substituted aryl, heteroaryl, substituted heteroaryl, aryloxy, substituted aryloxy, halogen, haloalkyl, cyano, nitro, nitrone, amino, amido, —C(O)H, —C(O)—, —C(O)O—, —S—, —S(O)₂, —OC(O)—O—, —NR—C(O), —NR—C(O)—NR, —OC(O)—NR, wherein R is H or lower alkyl, acyl, oxyacyl, carboxyl, carbamate, sulfonyl, sulfonamide, and sulfuryl.

As used herein, “alkenyl” refers to straight or branched chain hydrocarbyl groups having at least one carbon-carbon double bond, and having in the range of 2 up to about 500 carbon atoms, and “substituted alkenyl” refers to alkenyl groups further bearing one or more substituents as set forth above.

As used herein, “cycloalkyl” refers to cyclic ring-containing groups typically containing in the range of about 3 up to about 20 carbon atoms, and “substituted cycloalkyl” refers to cycloalkyl groups further bearing one or more substituents as set forth above.

As used herein, “aryl” refers to aromatic groups having in the range of 6 up to about 20 carbon atoms. “Substituted aryl” refers to aryl groups further bearing one or more substituents as set forth above. “Heteroaryl” refers to aryl groups having one or more heteroatoms (e.g., N, O, and S) as part of the ring structure.

As used herein, “heterocyclic” refers to cyclic (i.e., ring-containing) groups containing one or more heteroatoms (e.g., N, O, and S) as part of the ring structure, and having in the range of 3 up to about 14 carbon atoms and “substituted heterocyclic” refers to heterocyclic groups further bearing one or more substituents as set forth above. The term heterocyclic is also intended to refer to heteroaromatic moieties.

As used herein, “siloxane” refers to any compound containing a Si—O moiety. In certain embodiments, siloxanes of the invention include 2 or more repeating units of Si—O.

As used herein, the term “maleimido” refers to a compound bearing at least one moiety having the structure:

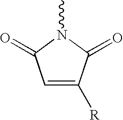

where R is H or lower alkyl.

“Imide” as used herein, refers to a functional group having two carbonyl groups bound to a primary amine or ammonia. The general formula of an imide of the invention is:

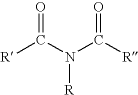

“Polyimides” are polymers of imide-containing monomers. Polyimides typically have one of two forms: linear or cyclic. Non-limiting examples of linear and cyclic (e.g. an aromatic heterocyclic polyimide) polyimides are shown below for illustrative purposes.

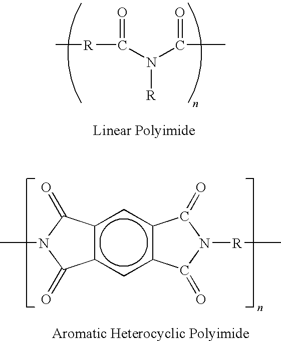

“Maleimide,” as used herein, refers to an N-substituted maleimide having the formula as shown below:

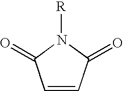

where the “R” group may be an aromatic, heteroaromatic, aliphatic, or polymeric moiety.

As used herein, the term “acrylate” refers to a compound bearing at least one moiety having the structure:

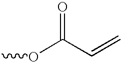

As used herein, the term “acrylamide” refers to a compound bearing at least one moiety having the structure:

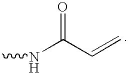

As used herein, the term “methacrylate” refers to a compound bearing at least one moiety having the structure:

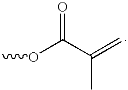

As used herein, the term “methacrylamide” refers to a compound bearing at least one moiety having the structure:

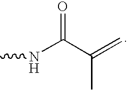

As used herein “epoxy” refers to a thermosetting epoxide polymer that cures by polymerization and crosslinking when mixed with a catalyzing agent or “hardener,” also referred to as a “curing agent” or “curative.” Epoxies of the present invention include, but are not limited to aliphatic, cycloaliphatic, glycidyl ether, glycidyl ester, glycidyl amine epoxies, and the like, and combinations thereof. Epoxies of the invention include compounds bearing at least one moiety having the structure:

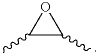

As used herein, the term “oxetane” refers to a compound bearing at least one moiety having the structure:

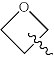

“Thermoplastic,” as used herein, refers to the ability of a compound, composition or other material (e.g. a plastic) to melt to a liquid when heated, and freeze to solid, often brittle and glassy, state when cooled sufficiently.

“Thermoset,” as used herein, refers to the ability of a compound, composition or other material to irreversibly “cure” to a stronger, harder form. Thermoset materials are typically polymers that may be cured, for example, through heat (e.g. above 200 degrees Celsius, or in the presence of appropriate catalysts at lower temperatures), via a chemical reaction (e.g. epoxy), or through irradiation (e.g. U.V. irradiation).

Thermoset materials, such as thermoset polymers or resins, are typically liquid or malleable forms prior to curing, and therefore may be molded or shaped into their final form, and/or used as adhesives. Curing transforms the thermoset resin into an infusible solid or rubber by a cross-linking process. Thus, energy and/or catalysts are added that cause the molecular chains to react at chemically active sites (unsaturated or epoxy sites, for example), linking the polymer chains into a rigid, 3-D structure. The cross-linking process forms molecules with a higher molecular weight and resultant higher melting point. During the reaction, when the molecular weight of the polymer has increased to a point such that the melting point is higher than the surrounding ambient temperature, the polymer becomes a solid material.

A “die” as used herein, refers to a small block of semiconducting material, on which a functional circuit is fabricated.

The acyl-containing moiety of curatives described herein can be varied considerably in the practice of the invention. Exemplary acyloxy (—OC(O)R) moieties according to Formula II are set forth below:

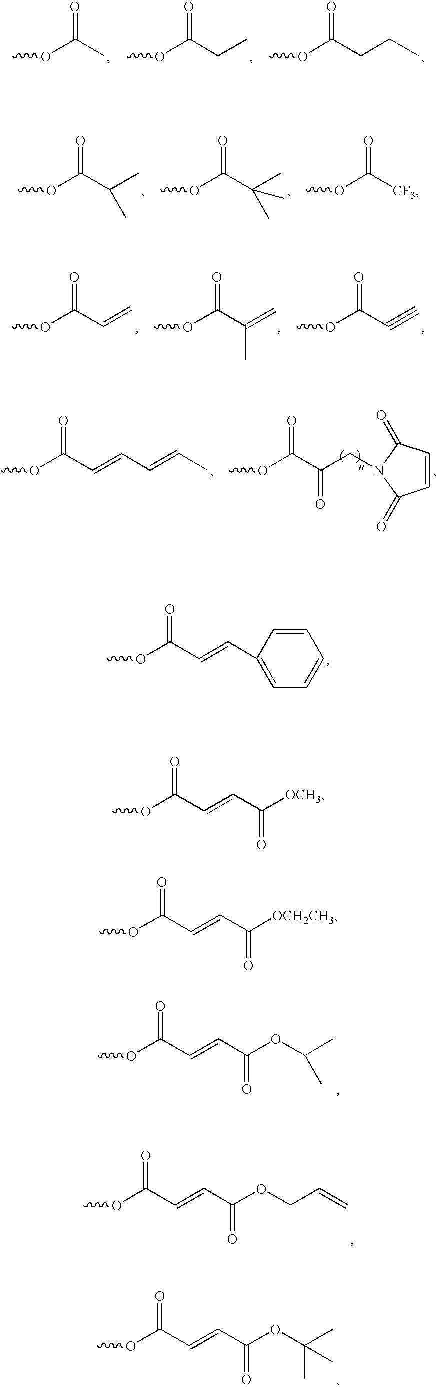

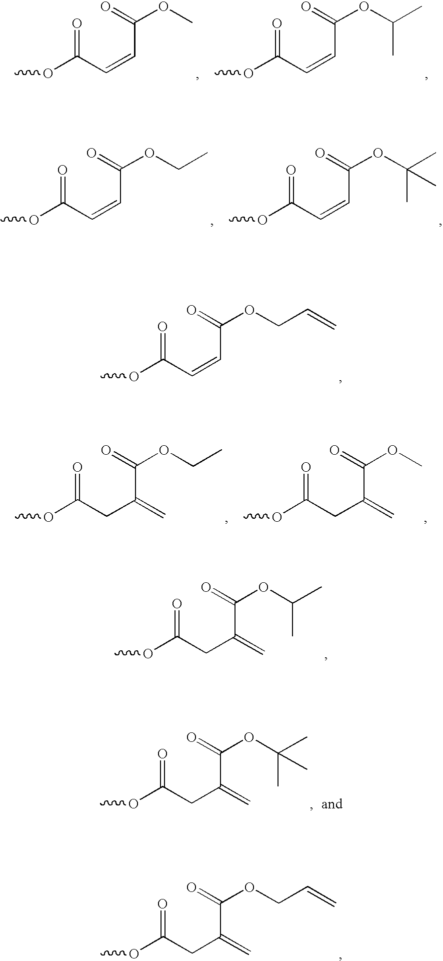

where n is 1 to 11.

Exemplary invention curing agents include:

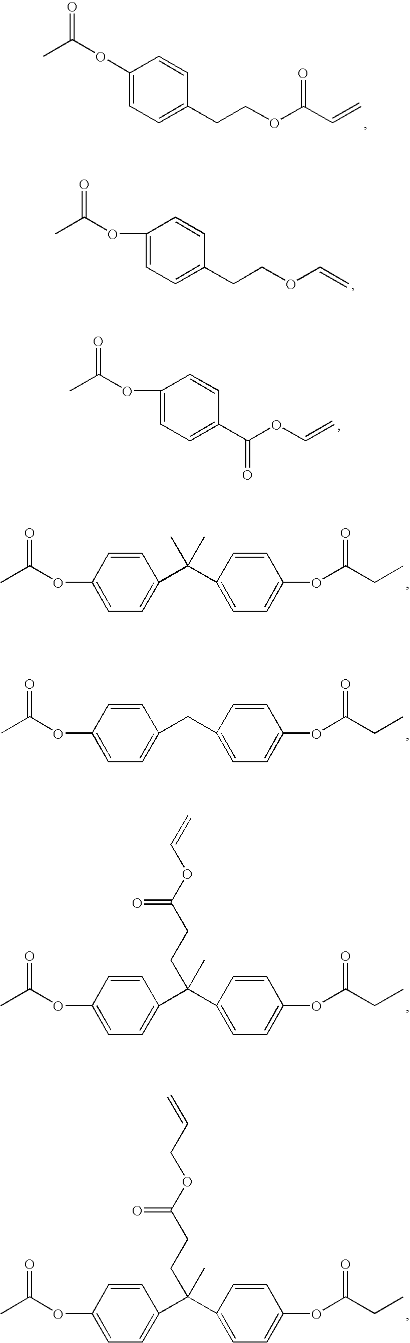

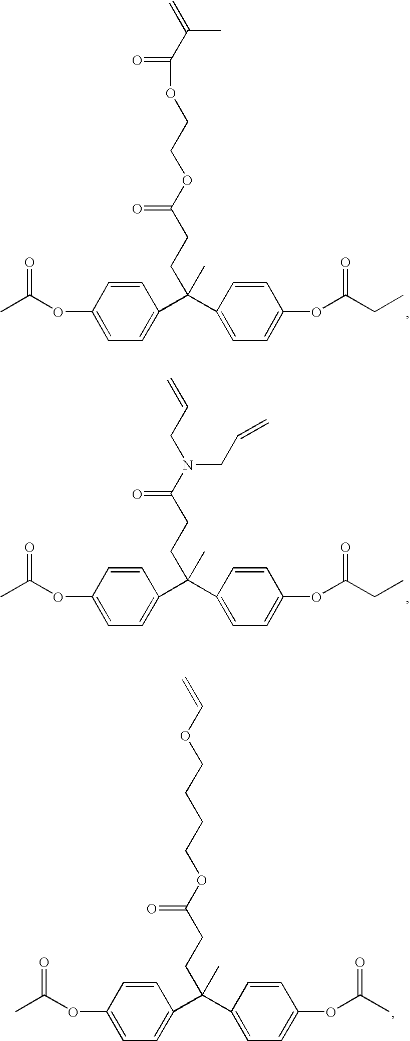

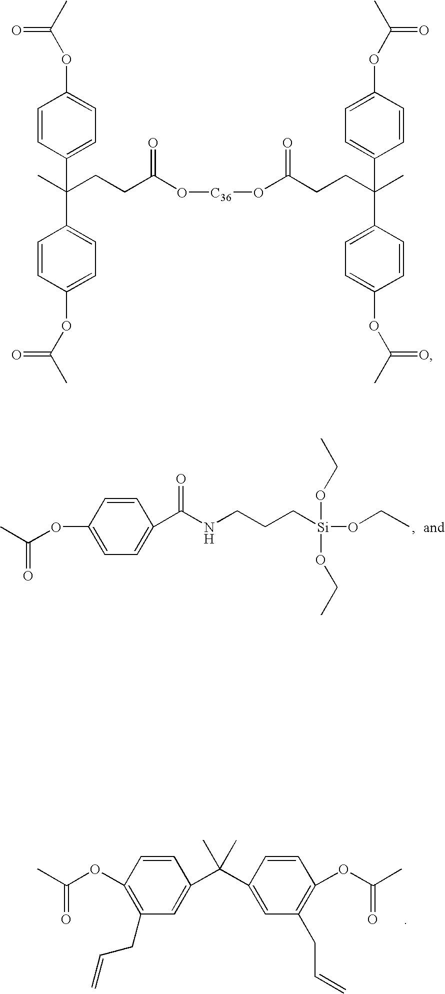

Dual functional acyloxy compounds of the present invention can be used to create new end-functionalized, monomers and oligomers through chain extension. Thus, according to one embodiment of the invention, a difunctional epoxy and a bisacyloxy compound can be reacted to form a linear oligomer. The oligomers can be chain terminated with a mono-acyloxy compound that also bears an independently polymerizable functional group. Where the end group is an acrylate, methacrylate, maleimide, citraconimide, diallylamide, styenyl, or other free radically polymerizable moiety, the oligomers can then be converted to a cross-linked thermoset in a second step. This dual stage cure is especially attractive for applications were it is desirable to apply an adhesive in liquid form, cure the material to a non-tacky thermoplastic state, and then cure this b-staged adhesive in a final heating step to bond two or more parts together.

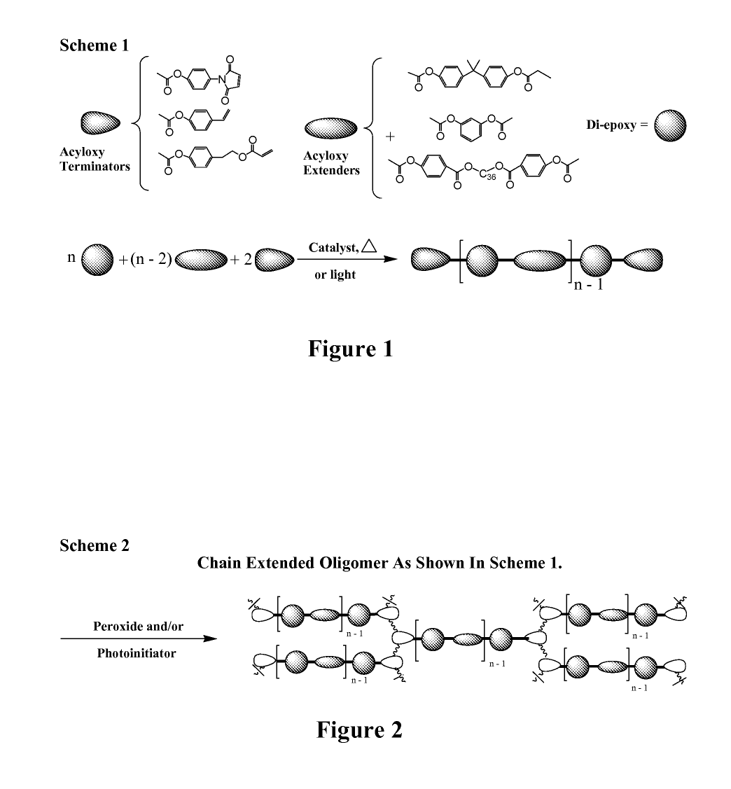

This dual stage cure method of the invention is particularly attractive for silicon wafer back coatings. The original mix of difunctional epoxies, difunctional acyloxy compounds, and suitably substituted mono-acyloxy compounds (along with coupling agents, catalysts, and optionally fillers) can be spin coated onto the back of a silicon wafer. The coating can then be b-staged with heat or light. The b-staging step can be represented by the chain extension and termination sequence shown in Scheme 1 (FIG. 1). The coated wafers can then be diced to yield individual microelectronic components, which may be thermally attached directly to a substrate, and/or stacked together. The thermal “tacking step” re-liquifies the oligomeric coating and provides a thermoplastic bond between the parts. The final bonding step involves a thermal (or in some cases light-based) cure to cross-link the b-staged functional oligomers as shown in Scheme 2 (FIG. 2). This method of assembly is highly desirable because it is easier to manufacture (especially for stacked die) than a traditional liquid adhesive assembly, and is much less expensive and wasteful compared to film-based adhesive technology.

Poly-acyloxy curatives are also contemplated for use in the practice of the invention. These are especially suited for pre-applied and/or film applications. Indeed, any novolak can be converted to a poly-acyloxy compound, and therefore a vast array of poly-acyloxy curatives are contemplated, including but not limited to those shown below.

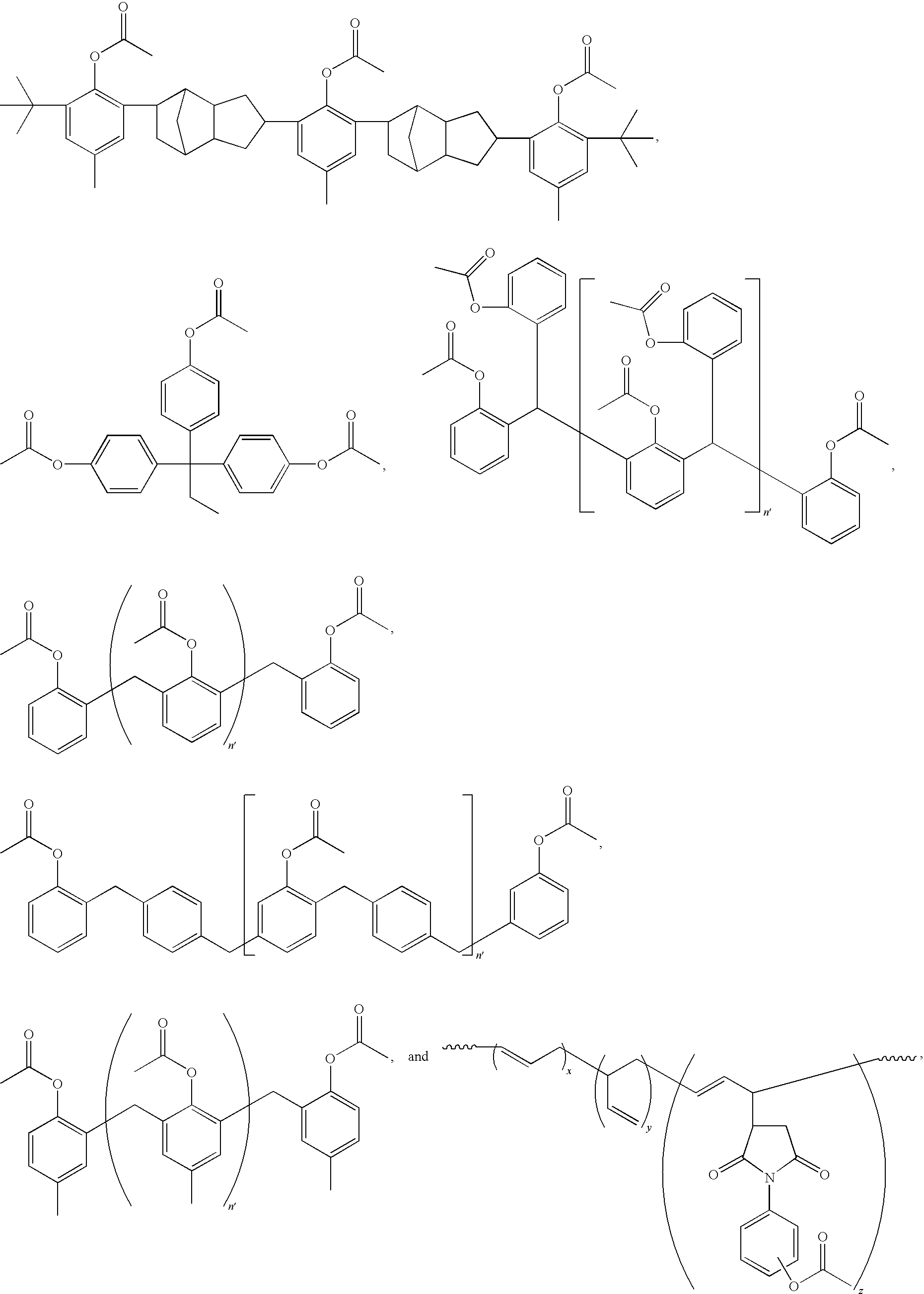

where n′ is 0 to about 10; x is 4 to about 50; y is 4 to about 50; and z is 2 to about 40.

Referring now to Formula II, the substituent R can be varied considerably in the practice of the invention. Exemplary N-acyl moieties include but are not limited to:

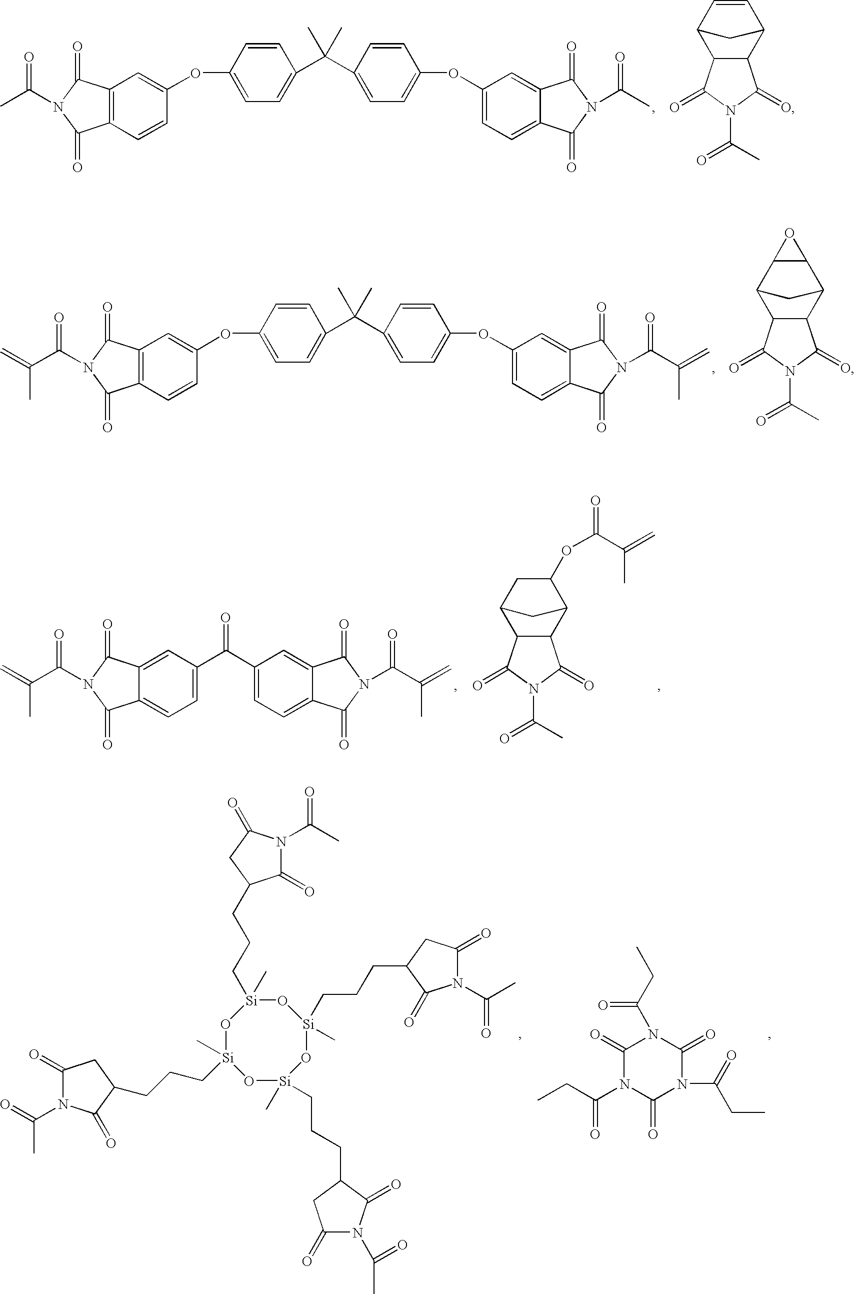

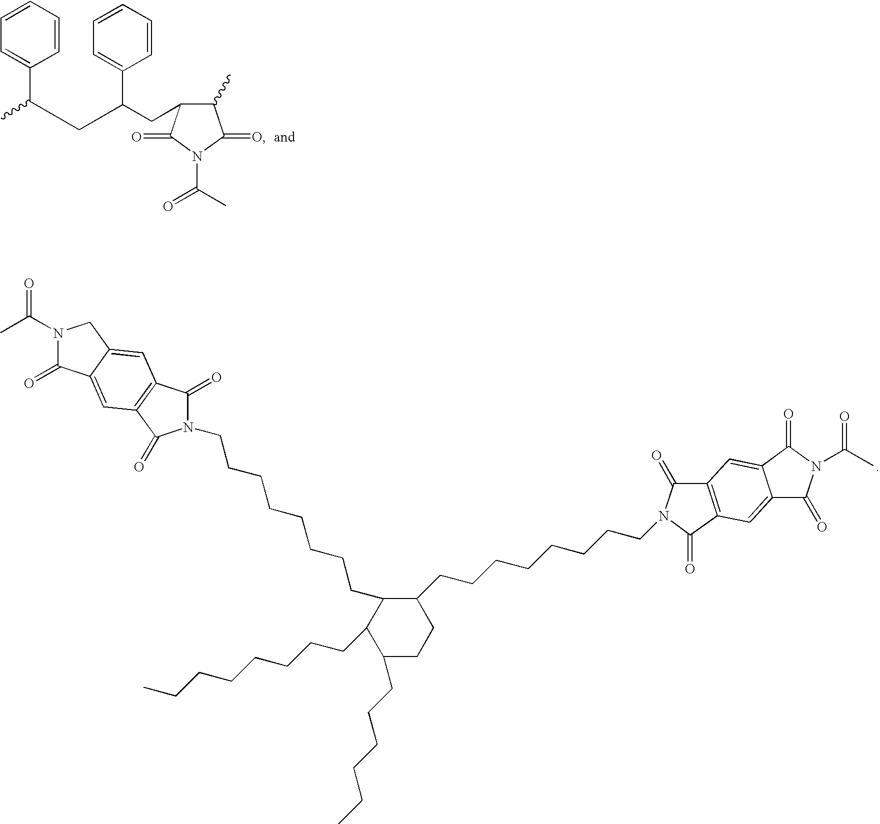

Additional exemplary invention curing agents are set forth below:

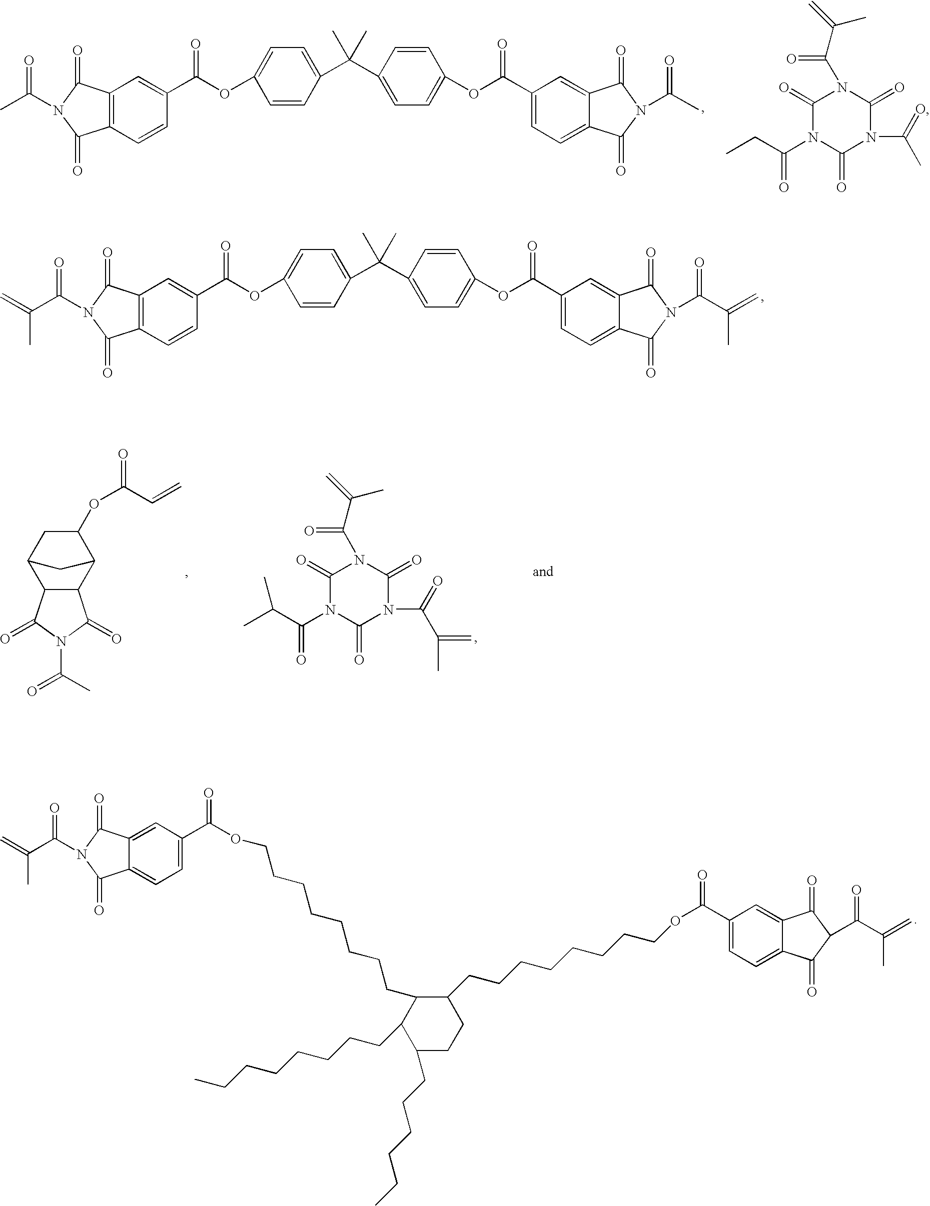

Poly-N-acyl curatives are also contemplated for use in the practice of the invention. These are especially suited for pre-applied and/or film applications. Indeed, any polymer containing anhydride residues in the main-chain or grafted to the backbone can be converted to a poly-N-acyl compound, and therefore a vast array of poly-N-acyl curatives are contemplated, including, but not limited to those illustrated below.

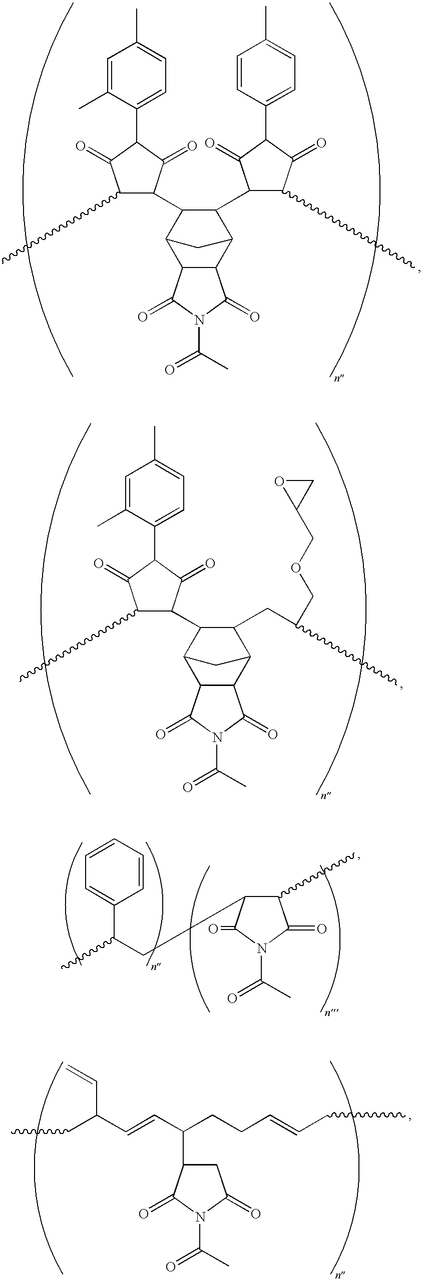

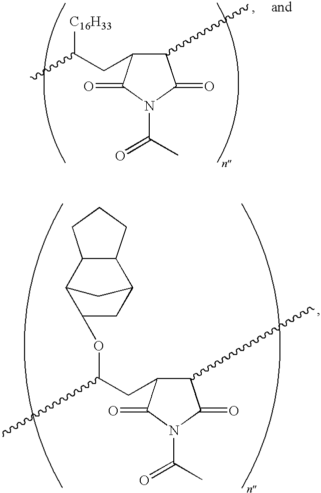

where each n″ and n′″ is independently 1 to about 10.

The compounds set forth below provide representative, non-limiting examples of phenyl acyloxy derivatives that have additional useful functionality. In some cases these compounds can be used to make high Tg, linear, segments within a thermoset (i.e. where the molecule bears both epoxy and acyloxy functionality). The maleimide functional compounds can be used to make polymaleimides in situ, which would be available to participate in the rich cure chemistry of polymaleimides (ene/Diels-Alder, Michael addition, free-radical, etc.). It should be noted that most of these compounds are shown as their phenyl acetates, however any of the previous acyloxy moieties are contemplated for use in this embodiment of the invention. In some embodiments, the isopropenyl compounds could be used for ene/Diels-Alder cures of BMIs.

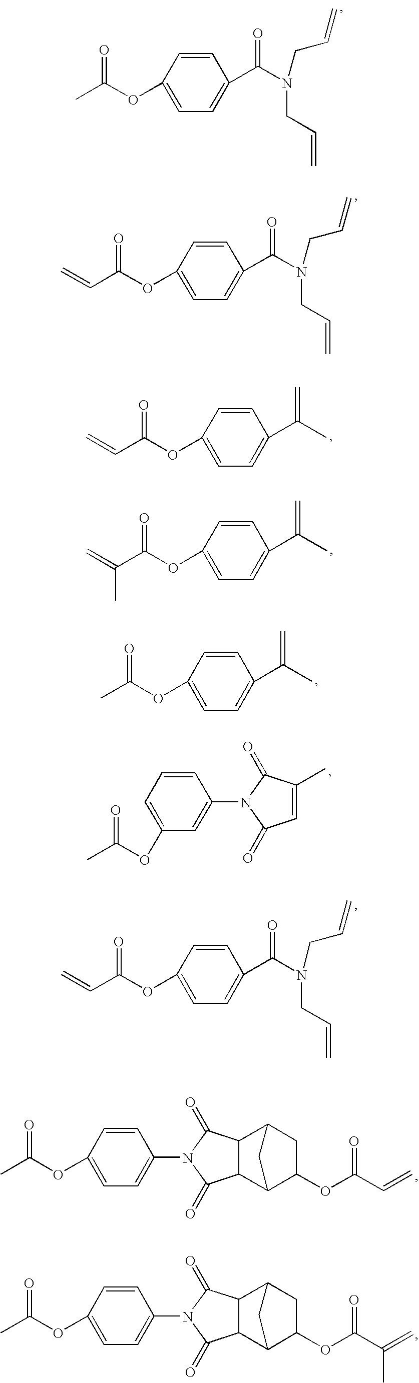

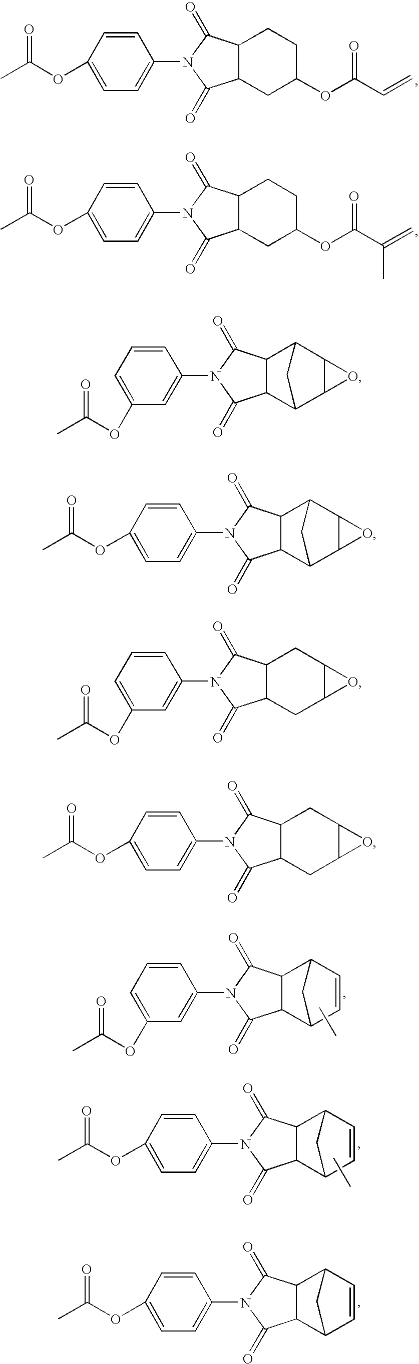

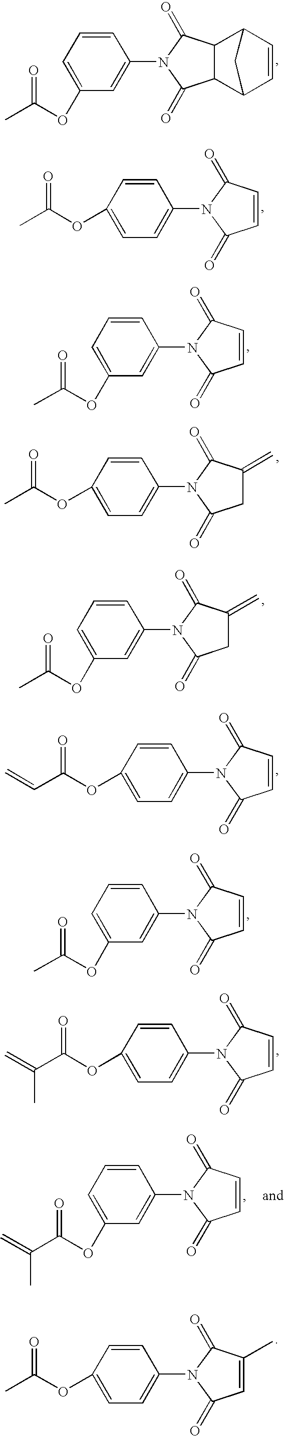

The compounds set forth below are liquids and would therefore be suited for use in paste based adhesives.

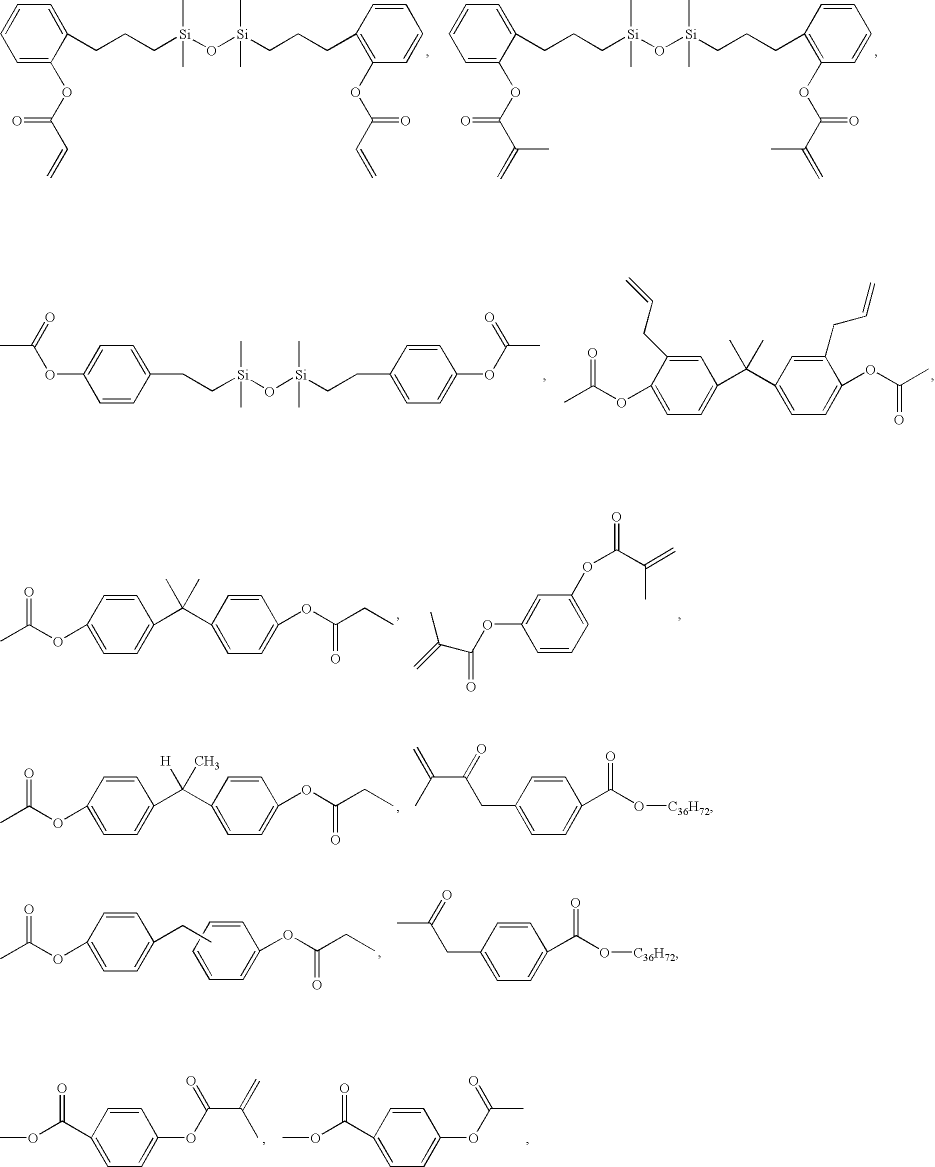

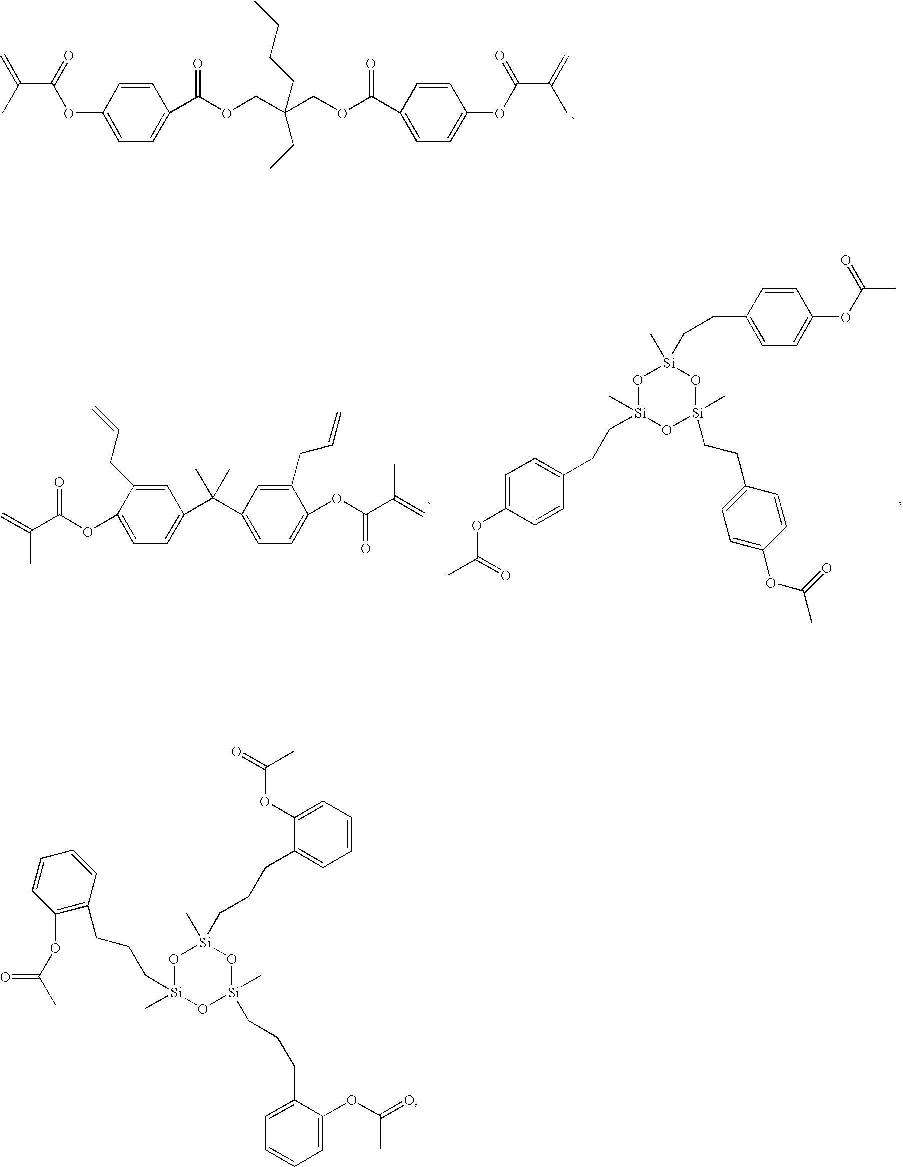

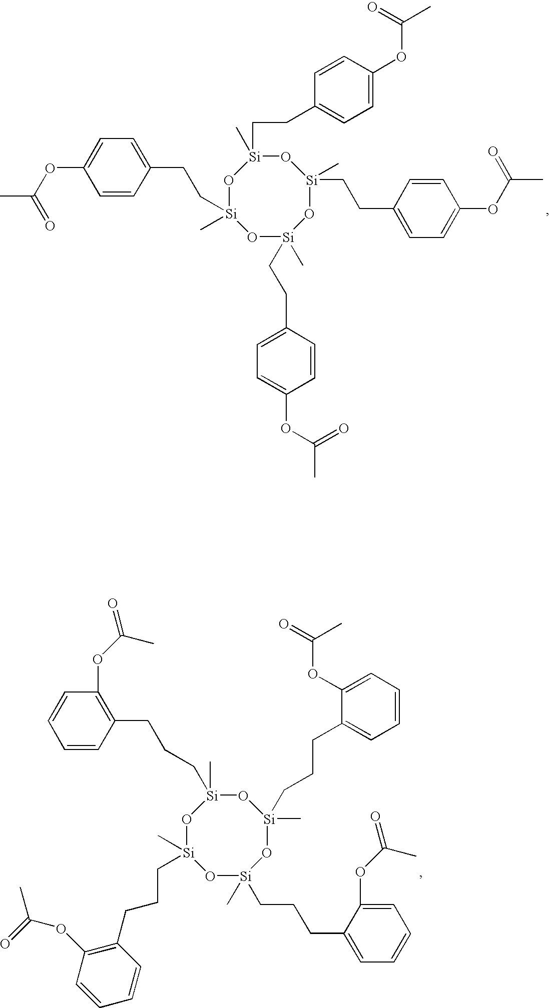

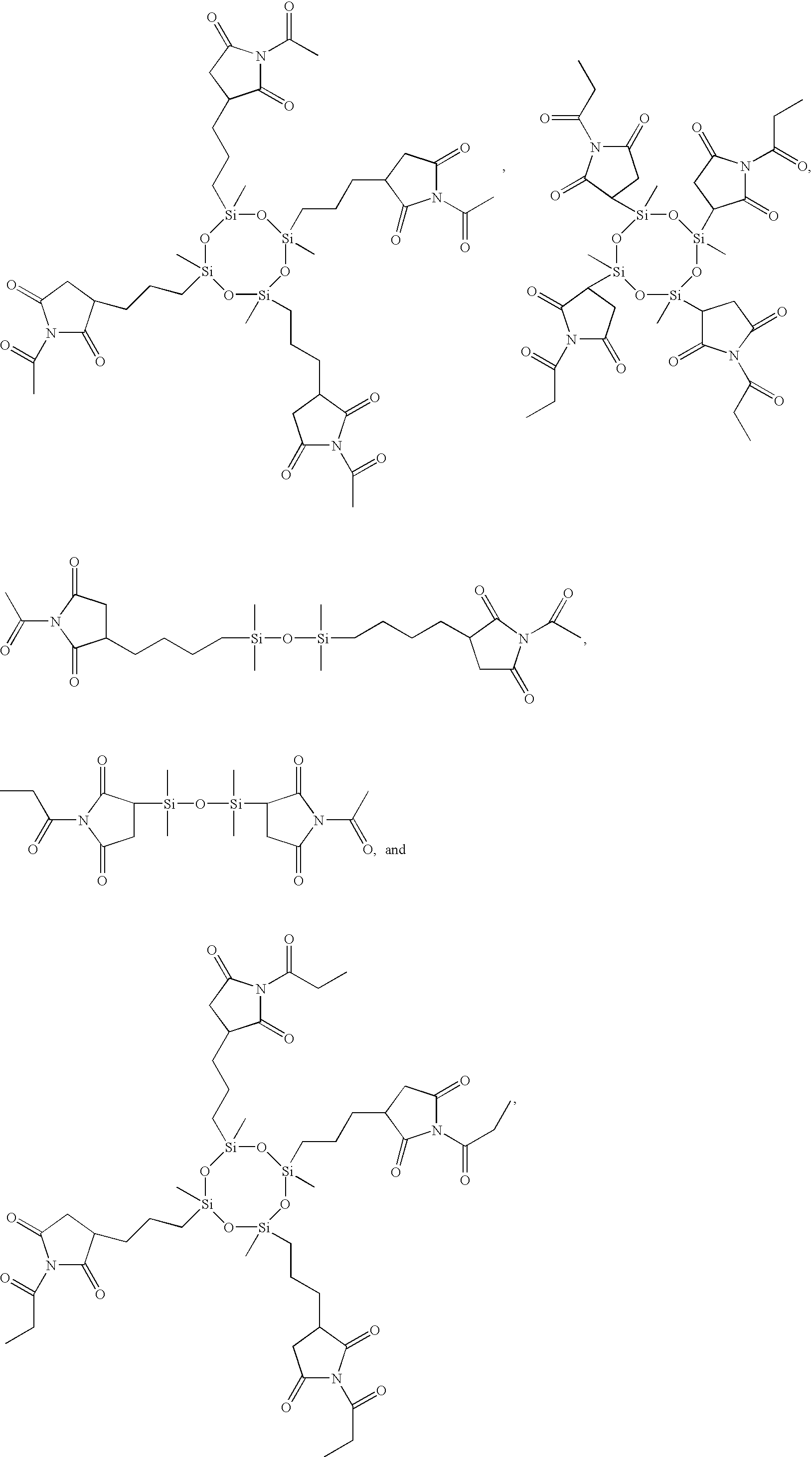

Epoxy resins contemplated for use in the practice of the invention include, but are not limited to, aliphatic, cycloaliphatic, glycidyl ether, glycidyl ester, glycidyl amine epoxies.

Glycidyl ether epoxy resins contemplated for use in the practice of the invention include, but are not limited to, a glycidyl ether of a phenol, an amine, an alcohol, or an isocyanurate, such as a phenyl glycidyl ether, a cresol glycidyl ether, a nonylphenyl glycidyl ether, and a p-tert-butylphenyl glycidyl ether; a diglycidyl ether or a trisglycidyl ether of a phenolic compound such as bisphenol A, bisphenol F, ethylidinebisphenol, dihydroxydiphenyl ether, N,N′-disalicylal-ethylenediamine, triglycidyl-p-aminophenol, N,N,N′,N′-tetraglycidyl-4,4′-diphenylmethane, triglycidyl isocyanurate, bis(4-hydroxyphenyl)sulfone, bis(hydroxyphenyl)sulfide, 1,1-bis(hydroxyphenyl)cyclohexane, 9,19-bis(4-hydroxyphenyl)fluorene, 1,1,1-tris(hydroxyphenyl)ethane, tetrakis(4-hydroxyphenyl)ethane, trihydroxytritylmethane, 4,4′-(1-alpha-methylbenzylidene)bisphenol, 4,4′-(1,3-componentthylethylene)diphenol, componentthylstilbesterol, 4,4′-dihyroxybenzophenone, resorcinol, catechol, and tetrahydroxydiphenyl sulfide; a glycidyl ether of a cresol formaldehyde condensate; a glycidyl ether of a phenol formaldehyde condensate; a glycidyl ether of a cresol dicyclopentadiene addition compound; a glycidyl ether of a phenol dicyclopentadiene addition compound; a glycidyl ether of a fused ring polyaromatic phenol such as dihydroxy naphthalene, 2,2′-dihydroxy-6,6′-dinaphthyl disulfide, and 1,8,9-trihydroxyanthracene; a glycidyl ether of an aliphatic alcohol such as a diglycidyl ether of 1,4 butanediol, a diglycidyl ether of diethylene glycol, a diglycidyl ether of neopentyl glycol, a diglycidyl ether of cyclohexane dimethanol, a diglycidyl ether of tricyclodecane dimethanol, a trimethyolethane triglycidyl ether, and a trimethyol propane triglycidyl ether; a glycidyl ether of a polyglycol such as Heloxy 84™, Heloxy 32™, a polyglycidyl ether of castor oil, and a polyoxypropylene diglycidyl ether; a glycidyl derivative of an aromatic amine; ester linked epoxies, such as Heloxy 71 and glycidyl methacrylate. Other glycidyl ether epoxies contemplated herein include homo- and co-polymers based on allyl glycidyl ether.

Cycloaliphatic epoxy compounds contemplated for use in the practice of the invention include, but are not limited to, cyclohexene oxide; 3-vinylcyclohexene oxide; vinylcyclohexene dioxide; dicylcopentadiene dioxide; tricyclopentadiene dioxide; tetracyclopentadiene dioxide; norbornadiene dioxide; bis(2,3-epoxycyclopentyl)ether; limonene dioxide; 3′,4′-epoxycyclohexamethyl-3,4-epoxycyclohexanecarboxylate; 3,4-epoxycyclohexyloxirane; 2(3′,4′-epoxycyclohexyl)-5,1″-spiro-3″,4″-epoxycyclohexane-1,3-dioxane; bis(3,4-epoxycyclohexamethyl)adipate; and the like.

Aliphatic epoxy compounds contemplated for use in the practice of the invention include, but are not limited to, epoxidized polybutadiene; epoxidized polyisoprene; epoxidized poly(1,3-butadiene-acrylonitrile); epoxized soybean oil; epoxidized castor oil; dimethylpentane dioxide; divinylbenzene dioxide; butadiene dioxide; and 1,7-octadiene dioxide.

As used herein, “b-stageable” means that the adhesive has a first solid phase followed by a tacky rubbery stage at elevated temperature, followed by yet another solid phase at an even higher temperature. The transition from the tacky rubbery stage to the second solid phase is thermosetting. However, prior to that, the material behaves similarly to a thermoplastic material. Thus, such adhesives allows for low lamination temperatures while providing high thermal stability.

The b-stageable adhesive can be dispensed onto a die or a substrate by a variety of methods well known to those skilled in the art. In some embodiments, the adhesive is cast from solution using techniques such as spin coating, spray coating, stencil printing, screen printing, dispensing, and the like.

In certain embodiments, a solvent may be employed in the practice of the invention. For example, when the b-stageable adhesive is spin coated onto a circular wafer, it is desirable to have an even coating throughout the entire wafer, i.e., the solvent or solvent system should have the ability to deliver the same amount of adhesive to each point on the wafer. Thus, the adhesive will be evenly coated throughout, i.e., there will be the same amount of material at the center of the wafer as at the edges. Ideally, the adhesive is “Newtonian”, with a thixotropic slope of 1.0. In certain embodiments, the solvent or solvent systems used to dispense the b-stageable adhesive have thixotropic slopes ranging from 1.0 to about 5.

In some instances, the b-stageable adhesive is dispensed onto the backside of a die that has been optionally coated with a polyimide. To achieve this goal, in certain embodiments, the solvent system will include a polar solvent in combination with a non-polar solvent. In addition, the polar solvent typically has a lower boiling point than the non-polar solvent. Without wishing to be limited to a particular theory, it is believed that when the adhesive is dispensed and then b-staged, the lower boiling polar solvent escapes first, leaving behind only the non-polar solvent, essentially precipitating the polymer uniformly.

In some embodiments, the solvent or solvent system has a boiling point ranging from about 150° C. up to about 300° C. In some embodiments, the solvent system is a combination of dimethyl phthalate (DMP), NOPAR 13, and terpineol. In other embodiments, the solvent system is a 1:1 (by volume) ratio of terpineol and NOPAR 13.

Fillers contemplated for use in the practice of the present invention can be electrically conductive and/or thermally conductive, and/or fillers which act primarily to modify the rheology of the resulting composition. Examples of suitable electrically conductive fillers which can be employed in the practice of the present invention include silver, nickel, copper, aluminum, palladium, gold, graphite, metal-coated graphite (e.g., nickel-coated graphite, copper-coated graphite, and the like), and the like. Examples of suitable thermally conductive fillers which can be employed in the practice of the present invention include graphite, aluminum nitride, silicon carbide, boron nitride, diamond dust, alumina, and the like. Compounds, which act primarily to modify rheology, include polysiloxanes, silica, fumed silica, fumed alumina, fumed titanium dioxide, calcium carbonate, and the like.

The acyloxy curatives described in this invention can be prepared through a variety of methods known in the art. These synthetic methods include, but are not limited to, the reaction of phenolic compounds with carboxylic acid anhydrides, optionally in the presence of a catalyst. They can be prepared through the reaction of phenols with carboxylic acid chlorides. They may also be prepared via the condensation of phenols and carboxylic acids in the presence of a dehydrating agent, such as N,N′-dicyclohexylcarbodiimide.

The N-acyl curatives of this invention can also be prepared via a number of methods from the corresponding imides. These methods include all of those previously described for the preparation of acyloxy compounds. Thus, the N-acyl compounds may be prepared via the reaction of imides with carboxylic acid anhydrides, optionally in the presence of a catalyst. They can be prepared through the reaction of imides with carboxylic acid chlorides, optionally in the presence of a basic acid acceptor. They can also be made via the direct condensation of an imide and a carboxylic acid in the presence of a dehydrating agent.

The invention will now be further described with reference to the following non-limiting examples.

### EXAMPLES

It should be noted that for each of the following exemplary compounds, where the substitution on the backbone is asymmetric or where the molecule has been extended with another bi-functional reactant, that only a single representative structure is shown. That is to say, such compounds are in fact composed of statistical distributions of several molecules. Only the most predominant species in these distributions are shown.

### Example 1

### Preparation of a Phenol Functional Curative

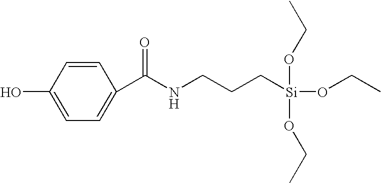

A 500 mL, 2-neck flask was charged with 44.27 g (0.2 mole) 3-aminopropyltriethoxysilane, and 20.79 g (0.21 mole) butyl-4-hydroxybenzoate. The flask was equipped with a Dean-Stark trap condenser and bubbler. The mix was then stirred magnetically and heated at 170° C. under an argon blanket for 41.25 hours. Approximately 18.0 mL of butanol was collected in the trap (theoretical yield=18.3 mL). The mix was sparged with argon at 170° C. for forty-five minutes. The product was poured out of the container while still hot. It was a very viscous amber liquid at room temperature. A total of 65.6 g of product was recovered (96.0% of theoretical yield). An FTIR run on this compound had a broad —OH absorbance as well as strong absorptions at 2930, 1688, 1605, 1531, 1270, 1162, 1073, 953, 848, and 769 wavenumbers.

### Example 2

### Preparation of a Phenyl Acetate Curative

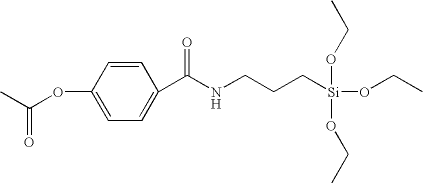

A portion of the compound from EXAMPLE 1 was converted to the phenyl acetate shown above. A 250 mL flask was charged with 37.15 g (0.11 mole) of the compound from EXAMPLE 1, 11.02 g (0.11 mole) acetic anhydride, and 0.1 g of dimethylaminopyridine. This mix was heated and stirred at 90° C. for two hours. The acetic acid side product was then removed via rotary evaporation and sparge. The final product weighted 40.5 g (97% of theoretical yield). An FTIR spectrum of this material revealed a small amide N—H stretch at 3318 along with prominent absorptions at 2934, 1760, 1639, 1501, 1268, 1198, 1073, 913, and 762 wavenumbers.

### Example 3

### Comparison of Epoxy Formulations Containing Phenol Functional Curative to the Corresponding Phenyl Acetate

The following example demonstrates the remarkably improved adhesion for an epoxy resin cured using an acyloxy coupling agent from EXAMPLE 2, versus the analogous phenol-functional coupling agent from EXAMPLE 1, which does not contain the acyloxy moiety.

TABLE 1
Properties of Expoxy Formulations Containing a Phenol Functional
Curative and Corresponding Phenyl Acetate

| | Formulation 1 | Formulation 2 |
|---|---|---|
| Composition | | |
| Tactix 756 epoxy | 31.6% | 31.6% |
| Ricon | 15.2% | 15.2% |
| Terpineol | 36.7% | 36.7% |
| Curezol 2MA | 1.1% | 1.1% |
| Silica | 1.1% | 1.1% |
| EXAMPLE 1 compound | 2.1% | 0.0 |
| EXAMPLE 2 compound | 0.0 | 2.1% |
| Adhesion* | | |
| (300 × 300 Si on ceramic @ | 11.1 | 31.5 |
| 260° C. 175° C. | | |
| 60 min ramp cure + 4 hour | | |
| PMC) units are kg force. | | |

*The die-shear adhesion was measured as kg force on a Dage Series 4000.

The phenyl acetate functional coupling agent had almost three times the 260° C. adhesion of its phenol functional counterpart. Even at a relatively low percentage of the entire composition, the acyloxy compound is a superior epoxy curative compared to the free phenol.

### Example 4

### Preparation of Acrylic Acid 2-(4-Hydroxy-Phenyl)-Ethyl Ester Curative for Hybrid Epoxy and Free-Radical Cure Adhesives

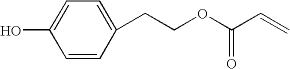

The compound shown above was designed for use as a possible hybrid monomer for adhesive compositions comprising epoxies and free-radical cure monomers. A 500 mL, two-neck flask was charged with 27.63 g (0.2 mole) 2-(4-hydroxyphenyl)ethyl alcohol, 150 mL toluene, 18.02 g (0.25 mole) acrylic acid, 40 mg hydroquinone, and 1.5 g methanesulfonic acid. The flask was equipped with a trap and condenser. The mixture was then refluxed under a mild air sparge for 1.5 hours. A total of 3.7 mL water (theoretical yield=3.6 mL) was collected in the trap. The mixture was then cooled and treated with 12 g sodium bicarbonate plus 3 g water until carbon dioxide evolution ceased. The mix was dried with 8 g magnesium sulfate and then passed over 15 g silica gel. The toluene was removed to yield 38.33 g (99.7% of theoretical yield) of a yellow liquid. The compound had prominent absorptions at 3394, 1699, 1635, 1614, 1514, 1408, 1264, 1196, 1059, 981, and 811 wavenumbers.

### Example 5

### Preparation of Acrylic Acid 2-(4-Acetoxy-Phenyl)-Ethyl Ester Curative for Hybrid Epoxy and Free-Radical Cure Adhesives

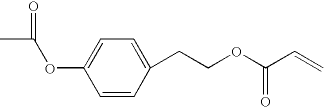

The phenyl acetate cousin of the compound from EXAMPLE 4 was prepared according to an identical procedure except that 20.42 g (0.2 mole) acetic anhydride was added after the initial acrylate esterification was complete. This mixture was stirred overnight at 60° C. Work-up afforded 46.44 g (99.1% of theoretical yield) of a light yellow, low viscosity liquid. The compound had prominent absorptions at 1755, 1724, 1635, 1509, 1497, 1369, 1181, 1058, 984, 909, and 809 wavenumbers.

### Example 6

### Comparison of Epoxy Formulations Containing Hydroxy and Acetoxy Curatives

Two weight percent dicumyl peroxide was added to each of the compounds from Examples 4 and 5. These mixtures were evaluated by DSC and TGA. The results of these tests are shown in the following table:

TABLE 2
Properties of Expoxy Formulations Containing Acrylic Acid 2-(4-
Hydroxy-Phenyl)-Ethyl Ester Curative or Corresponding Phenyl Acetate

| | | Cure Energy |
|---|---|---|
| Example (w/2% Diucp) | Retained Weight @ 300° C. | (J/g) |
| 4 | 40.1% | 6.6 |
| 5 | 89.7% | 223.8 |

The results in Table 2 indicate that the cure of the acrylate function for the EXAMPLE 4 compound was practically non-existent. This was also evident from the high weight loss for this example. The phenyl ester compound from EXAMPLE 5, by contrast, had a strong exotherm and almost 90% retained weight at 300° C. Capping the phenol with an ester function thus overcomes the inherent free-radical cure inhibition demonstrated by the original compound.

### Example 7

### Preparation of a Mixed Acetate Propionate of Bisphenol A

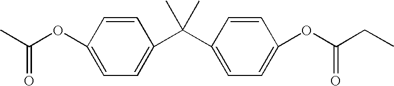

A 250 mL flask was charged with 45.66 g (0.2 mole) bisphenol A, 20.42 g (0.1 mole) acetic anhydride, 26.04 g (0.1 mole) propionic anhydride, and 0.1 g DMAP catalyst). This mixture was stirred in a bath maintained at 90° C. for 1.5 hours. The residual acetic and propionic acids were then stripped off to yield a colorless liquid that weighed 64.5 g (99% of theoretical yield). It should be noted that the above representation of the example compound represents about 50% of the total, while the remainder is approximately a one to one mix of the diacetate and dipropionate. An advantage of this mixed product is that it has a lower melting point than any of the individual components. The bisacyloxy compound had a 25° C., viscosity of 1,873 centipoise. An FTIR on this liquid showed prominent absorptions at 2971, 1756, 1504, 1367, 1166, 1015, 909, and 846 wavenumbers.

### Example 8

### Epoxy Generated from Mixed Acetate Propionate of Bisphenol A

A one to one equivalent mix of the diglycidyl ether of Bisphenol A (DER 332) and the bisacyloxy compound from EXAMPLE 7 was prepared. This mixture was catalyzed with two weight percent of DMAP. The cure of this mixture was analyzed via DSC and TGA. The cure (via DSC) was found to give a single symmetrical peak with an onset of 123° C., a peak maximum of 143° C. and a cure energy of 182 joules per gram. The mix had 98.82% retained weight at 300° C. and a decomposition onset (TGA, 10° C./min., air purge) of 420° C. These results suggest that the DMAP catalyzed cure of the bisacyloxy compound from EXAMPLE 7 was a very synergistic co-cure, where both the acyloxy and epoxy functions fully participated.

### Example 9

### Preparation of Diacetate of 2,2′-Diallylbisphenol A

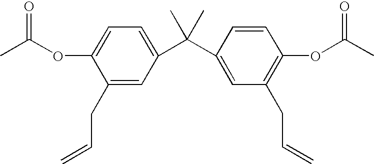

A 250 mL, single-neck flask was charged with 30.84 g (0.1 mole) o,o′-diallylbisphenol A, 20.42 g (0.2 mole) acetic anhydride, and 0.5 g DMAP. This mixture was stirred at 85° C. for one hour and the residual acetic acid was then removed to give 39.3 g (100% of theoretical yield) of a light orange liquid. The compound had prominent absorptions at 1759, 1495, 1367, 1197, 1117, 1008, 911, and 828 wavenumbers. The viscosity of this liquid was 2600 centipoise at 25° C. The viscosity of the o,o′-diallylbisphenol A starting material, by contrast, was 15,400 centipoise at the same temperature.

### Example 10

### Comparison of Acyloxy Curative with Corresponding Phenolic Curative

The following table shows the benefits of the acyloxy curative over a phenolic curative. The phenyl acetate of ortho diallyl bisphenol A phenol was synthesized. Both ortho diallyl bisphenol A phenol and the synthesized diphenyl acetate were use to compare the properties of both materials when cured with bisphenol A epoxy (DER 332 from Dow Chemical). Both materials were formulated as a 1:1 epoxy equivalent and two different catalysts were used for comparison. Anjicure PN-23 is a latent aliphatic amine catalyst and DMAP (N,N-dimethyaminopyridine) is a tertiary amine catalyst. Each catalyst was used at the level of 2% of the total resin.

The data shown in Table 3 below demonstrate the superior properties for the phenyl acetate curative in the terms of moisture absorption, adhesion, weight loss, cure energy, and viscosity. The ortho diallyl bis A phenol either had unacceptable worklife with the DMAP or cured to a thermoplastic under these conditions making it difficult to collect the TMA data.

TABLE 3
Properties of Epoxy Formulations Containing of Acyloxy Curative and
Corresponding Phenolic Curative

| | 115-51A | 115-51B | 115-51C | 115-51D |
|---|---|---|---|---|
| Phenol Acetate Experiments | | | | |
| 1:1 equivalents | | | | |
| DER 332 % | 48 | 48 | 54 | 54 |
| ortho diallyl Bis A phenol % | 52 | 52 | | |
| ortho diallyl Bis A phenylacetate % | | | 46 | 46 |
| Ajicure PN-23 (2% level) | X | | X | |
| DMAP (2% level) | | X | | X |
| Dynamic TGA (10° C./min) | | | | |
| Weight loss at 300° C. % | 1.1 | 2.1 | 0.5 | 1.2 |
| Onset for decompostion ° C. | 398 | 402 | 391 | 395 |
| DSC (10° C./min) | | | | |
| onset for cure ° C. | 85 | 50 | 75 | 75 |
| Peak cure temperature ° C. | 154 | 124 | 150 | 135 |
| Peak energy J/g | 155 | 107 | 187 | 178 |
| TMA | | | | |
| Alpha 1 | NA* | NA | 62 | 48 |
| Tg | NA | NA | 57 | 53 |
| Alpha 2 | NA | NA | 324 | 315 |
| RT modulus (70% Ag) | 4.5 GPa | NA | 6.5 GPa | 5.8 GPa |
| Viscosity 5 rpm (70% Ag) | 64 Kcps | cured | 18 Kcps | 18 Kcps |
| | | within | | |
| | | hours | | |
| 260° C. die shear | <1 kgf | | 2.7 kgf | 3.0 kgf |
| 150² mil dieBare Cu | | | | |
| Moisture Absorption | 1.28 | | 0.78 | 0.60 |
| 96 hours in 85/85% | | | | |

*Comments Thermoplastic TMA NA

### Example 11

### Preparation of the Bis-4-Acetoxybenzoate of Dimer Diol

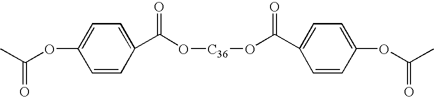

A one liter, single neck flask was charged with 55.25 g (0.4 mole) 4-hydroxybenzoic acid, 107.4 g (0.2 mole) dimer diol, 250 mL toluene, and 20 g of dry Amberlyst 46 resin. A magnetic stir bar was placed in the flask and a trap, condenser, and bubbler were attached. The mix was refluxed under an argon blanket for twenty-eight hours and 7.9 mL water (theoretical yield=7.2 mL) was collected. The Amberlyst catalyst was filtered out using a fitted funnel and the toluene was then removed. The product was then reacted with 40.84 g (0.4 mole) acetic anhydride plus 0.2 g DMAP at 90° C. for 1.5 hours. The acetic acid side product was then removed to yield 168.7 g (98% of theoretical yield) of a light yellow liquid. This compound had prominent infrared absorptions at 2922, 2853, 1763, 1720, 1271, 1190, 1158, 1115, 1016, and 912 wavenumbers.

### Experiment 12

### Epoxy Mixtures with Bis-4-Acetoxybenzoate of Dimer Diol

Mixtures were made using the DER 332 epoxy, a combination of two catalysts, and various levels of the curative from EXAMPLE 11. The mixture compositions are shown in Table 4 and the cured properties of those compositions are shown in Table 5.

TABLE 4
Epoxy + EXAMPLE 11 Curative Compositions

| Mixture | DER 332 % | Exp. 11 % | Anjicure PN23 % | Zn Undecylate % |
|---|---|---|---|---|
| A | 85 | 5 | 5 | 5 |
| B | 80 | 10 | 5 | 5 |
| C | 75 | 15 | 5 | 5 |
| D | 70 | 20 | 5 | 5 |

TABLE 5
Thermoset Cured Properties From Table 4 Mixtures

| | Alpha 1 (ppm/ | Alpha 2 (ppm/ | | Moisture |
|---|---|---|---|---|
| Mixture | ° C.) | ° C.) | T_{g} (° C.) | Uptake^{a} |
| A | 63.2 | 210 | 106.5 | 1.55 |
| B | 66.4 | 212 | 105.9 | 1.02 |
| C | 69.5 | 221 | 90.3 | 0.87 |
| D | 76.5 | 231 | 72.5 | 0.74 |

^{a}Percent weight gain at 85° C./85 RH over 168 hours

It is apparent from the results given in Table 5 that small additions of the curative from EXAMPLE 11 can dramatically reduce the moisture uptake, without significantly reducing the glass transition temperature or increasing the thermal expansion coefficient. Higher addition levels of this curative further reduced moisture uptake, but the thermoset cured property parameters were more severely impacted.

### Example 13

### Preparation of 2,7-dimethacryloxynapthalene

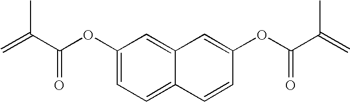

A 500 mL, single-neck flask was charged with 16.02 g (0.1 mole) 2,7-dihydroxynaphthalene, 150 mL toluene, 30.8 g (0.2 mole) methacrylic anhydride, 30 mg of BHT, and 0.5 g DMAP. This mixture was stirred on an oil bath set at 65° C. for 72 hours. The residual methacrylic acid was neutralized with 30 g sodium bicarbonate plus 5 g water, and then dried over 12 g anhydrous magnesium sulfate. The mixture was passed over 12 g of silica gel and the toluene was removed to yield 25.1 g (84.7% of theoretical yield) of what at first appeared to be a light red colored liquid. The compound converted to a waxy solid upon standing at room temperature. The product had significant infrared absorptions at 1730, 1637, 1316, 1202, 1114, 943, and 806 wavenumbers.

A portion of this compound was catalyzed with two weight percent of dicumyl peroxide. This mixture was found to have a cure onset of 137.4° C., and a cure maxima of 148.5° C. by DSC. The mix was found to have 93.9% retained weight at 300° C., and a decomposition onset of 423° C. (10° C./minute, air purge) via TGA. A cured sample of this compound was found to have a remarkably low alpha 1 value of 41.4 ppm/° C., an alpha 2 of 117 ppm/° C. and a T_{g} of 78.1° C. by TMA. This compound is a useful acyloxy curative. It can be used as a chain extender for di-functional epoxies. The extended, thermoplastic oligomer can be cross-linked through the pendant methacrylate moieties in a secondary free radical cure.

### Example 14

### Preparation of Acyloxy-Phenylmaleimide Mixture

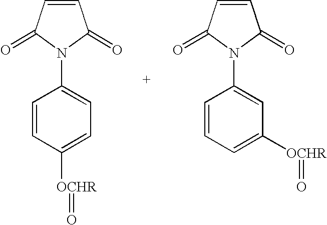 [0116] where R is CH₃ or CH₂CH₃

A 125 mL flask was charged with 1.89 g (0.01 mole) of 4-hydroxyphenylmaleimide, 1.89 g (0.01 mole) of 3-hydroxyphenylmaleimide and half an equivalent each of acetic anhydride and propionic anhydride along with about 10 mg of DMAP catalyst. The flask was stirred on a rotovap for two hours at 90° C. and then the residual acetic and propionic acids were removed by sparging. The resulting red liquid set up to an orange solid at room temperature. The product had strong infrared absorptions at 1756, 1717, 1510, 1398, 1196, 1148, 828, and 689 wavenumbers. The mixed compound was found to have a broad melting point via DSC. The melt onset was 93.8° C., with a melt minima at 107.9° C. The acyloxy-phenylmaleimide mixture appeared to be readily soluble in other monomers.

### Example 15

### Preparation of a Mixed N-Acylimide Curative

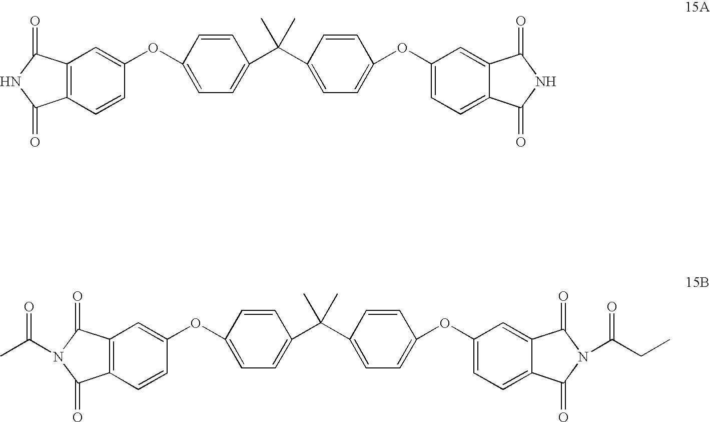

The imide precursor 15A was prepared from the commercially available Ultem BPADA (GE Plastics, Pittsfield, Mass.). Thus, 52.0 g (0.1 mole) of the dianhydride and 6.0 g (0.1 mole) urea were ground together in a mortar and pestle. This mixture was transferred to a single neck, 500 mL flask. The flask was equipped with a condenser and bubbler, and then heated in an oil bath that was controlled at 133° C. The mix foamed up as carbon dioxide and then water were evolved. The contents were occasionally stirred to insure homogeneity. The temperature bath was raised to and held at 165° C. for thirty minutes once CO₂ generation had ceased. The mix was cooled to room temperature and then 60 mL of deionized water was added. The slurry was transferred to a Buchner funnel and the solids were rinsed with deionized water. The solid was dried at 100° C. in an oven to yield 49.9 g (96.2% of theoretical yield) of a cream colored powder. An FTIR was run on this compound and it was found to have significant absorptions at 3264, 1766, 1716, 1598, 1476, 1361, 1237, 1041, 835, and 749 wavenumbers.

The mixed N-acyl curative 15B was prepared by charging a 250 mL, one-neck flask with 25.93 g (0.05 mole) compound 15A, 5.3 g (0.052 mole) acetic anhydride, 6.76 g (0.052 mole) propionic anhydride, 0.2 g DMAP catalyst, and 100 mL toluene. A magnetic stir bar was added and a condenser attached to the flask. This mixture was gently refluxed for twenty hours (during which time all of the solids went into solution). A light yellow solid precipitated out when the solution was cooled to room temperature. This solid was transferred to a Buchner funnel and rinsed with toluene. The solid was dried to yield 30.96 g (100% of theoretical yield) of a yellow-white powder. This compound was found to have a melting point of 197-200° C. An FTIR on the compound revealed significant absorptions at 2921, 1795, 1753, 1714, 1598, 1471, 1360, 1280, 1230, 1170, 1079, 840, and 745 wavenumbers.

### Example 16

### Preparation of an N-Acetylimide Curative Oligomer

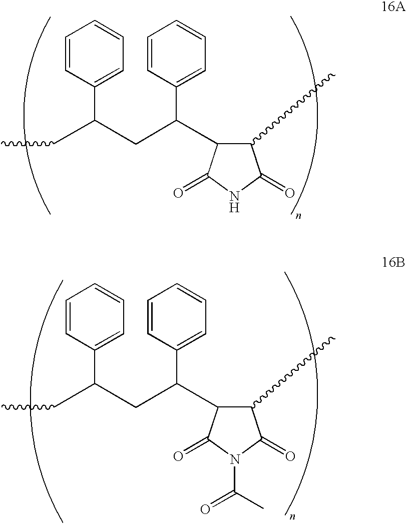

The imide precursor 16A was prepared from the commercially available poly(styrene-co-maleic anhydride) compound SMA-2000P (Sartomer Company, Inc. Exton Pa., USA). Thus, 30.8 g (0.1 equivalent) of the polyanhydride and 6.0 g (0.1 mole) urea were ground together in a mortar and pestle. This mixture was transferred to a single neck, 500 mL flask and 15 mL of NMP was added. The flask was equipped with a condenser and bubbler, and then heated in an oil bath that was controlled at 135° C. The mix foamed up as carbon dioxide and then water were evolved. The contents were occasionally stirred to insure homogeneity. The temperature bath was raised to and held at 165° C. for three hours once CO₂ generation had ceased. The mix was cooled to room temperature and then dissolved in 60 mL of acetone. The solution was dripped into 500 mL of vigorously stirred deionized water. The solid was collected and dried at 80° C. in an oven to yield 29.86 g (97.1% of theoretical yield) of a cream colored powder. An FTIR was run on this compound and it was found to have significant absorptions at 3207, 1771, 1711, 1453, 1381, 1181, 760, and 701 wavenumbers.

The N-acetyl curative oligomer 16B was prepared by slowly dripping 6.3 g (0.08 mole) acetyl chloride into a magnetically stirred solution containing 23.1 g (0.075 equivalents) 16A, 9.1 g (0.09 mole) triethylamine and 50 mL acetone. There was an immediate exotherm and a solid precipitate of triethylamine hydrochloride was observed to form. This mixture was stirred for another forty-five minutes and was then dripped into a one-liter beaker containing 500 mL of vigorously stirred deionized water. The solid was collected and then re-dissolved in 75 mL fresh acetone and the product was once again precipitated into 500 mL of deionized water. The solid was recovered via filtration and dried in an oven at 75° C. The product was an off-white fine powdered solid that weighed 24.57 g (93.7% of theoretical yield). An FTIR on the compound revealed significant absorptions at 3028, 2925, 1801, 1751, 1708, 1601, 1494, 1453, 1384, 1295, 1195, 759, and 704 wavenumbers.

### Example 17

### Epoxy Blends with the N-Acetylimide Curative Oligomer

A mixture was made that contained 70% by weight compound 16B, 30% limonene dioxide and one part per hundred of DMAP catalyst. A DSC was run on this composition and an exotherm was observed to occur with an onset of 153° C., a cure maximum of 170.7° C. and with a cure energy of 68 J/g.

Another mix was made consisting of 75% by weight compound 16B, 25% ERL-4221 (Dow Chemical) and one part per hundred DMAP catalyst. A DSC was run on this composition and an exotherm was observed with an onset of 121.6° C., a maximum at 169.2° C. and a cure energy of 48.2 J/g.

The limonene dioxide is a mixed cycloaliphatic and aliphatic epoxy compound while the ERL-4221 is a bi-functional cycloaliphatic epoxy. The 16B was shown to be an active curative for both of these epoxy compounds.

While this invention has been described with respect to these specific examples, it should be clear that other modifications and variations would be possible without departing from the spirit of this invention.

1. A curative for epoxy or oxetane resins having the structure of Formula I or Formula II: 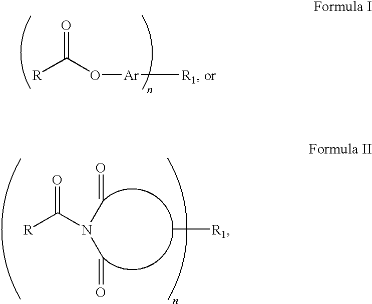
wherein R and R₁ are each independently substituted or unsubstituted aliphatic, heteroaliphatic, aromatic, heteroaromatic, siloxane, maleimido, or cinnamyl;
Ar is substituted or unsubstituted aryl or hetero-aryl having from 6 to about 20 carbon atoms; and
n is 1 to about 11.

2. The curative of claim 1, wherein R and R₁ are each independently substituted or unsubstituted alkyl, cycloalkyl, alkenyl, aryl, or heterocyclic.

3. The curative of claim 1, wherein Ar is substituted or unsubstituted C₆ to about C₁₁ aryl or heteroaryl.

4. The curative of claim 1, wherein n is 1 to about 6.

5. The curative of claim 1, wherein the curative is a liquid at room temperature.

6. The curative of claim 1, selected from:

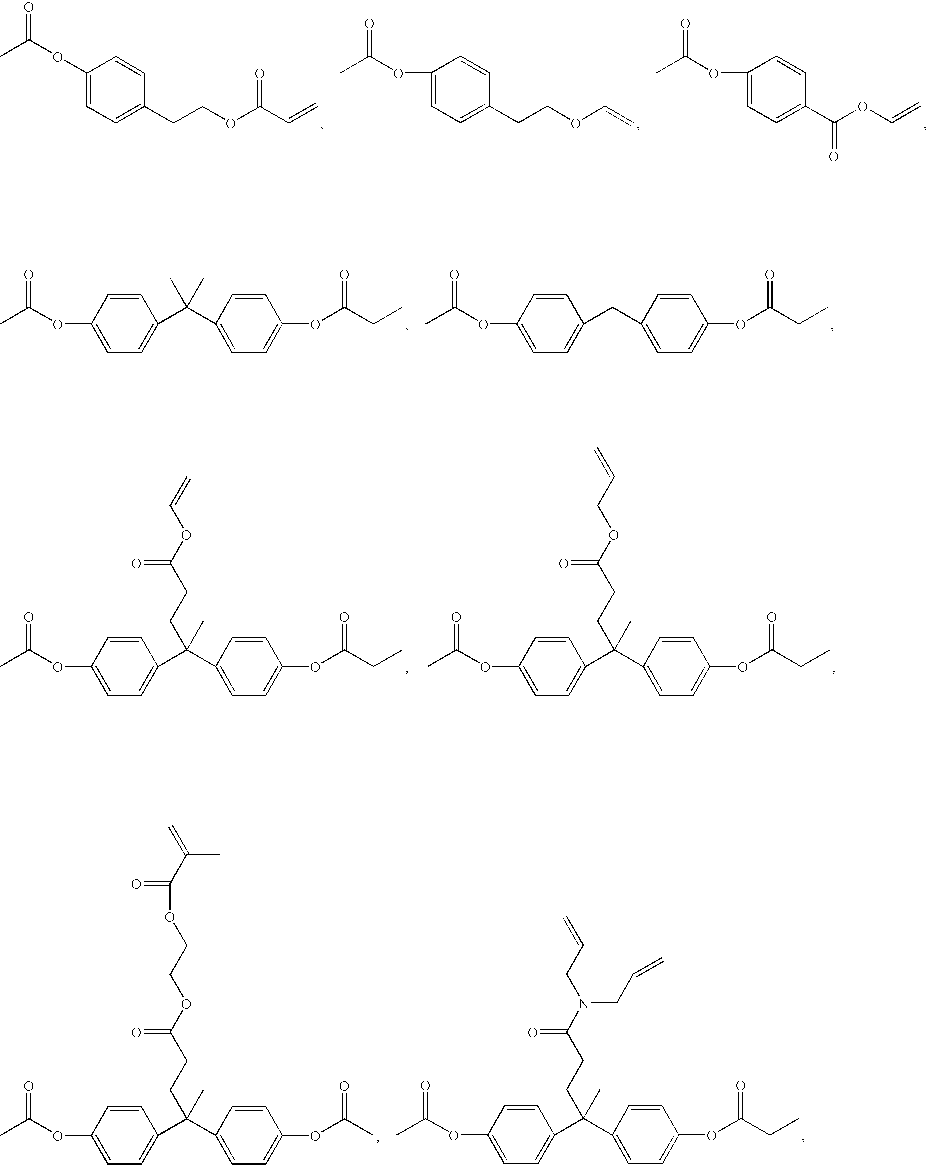

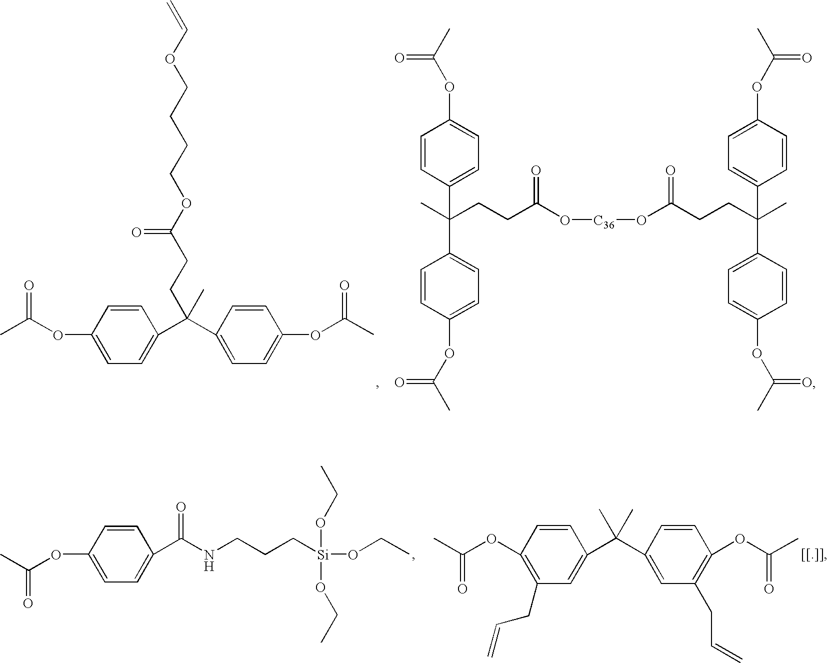

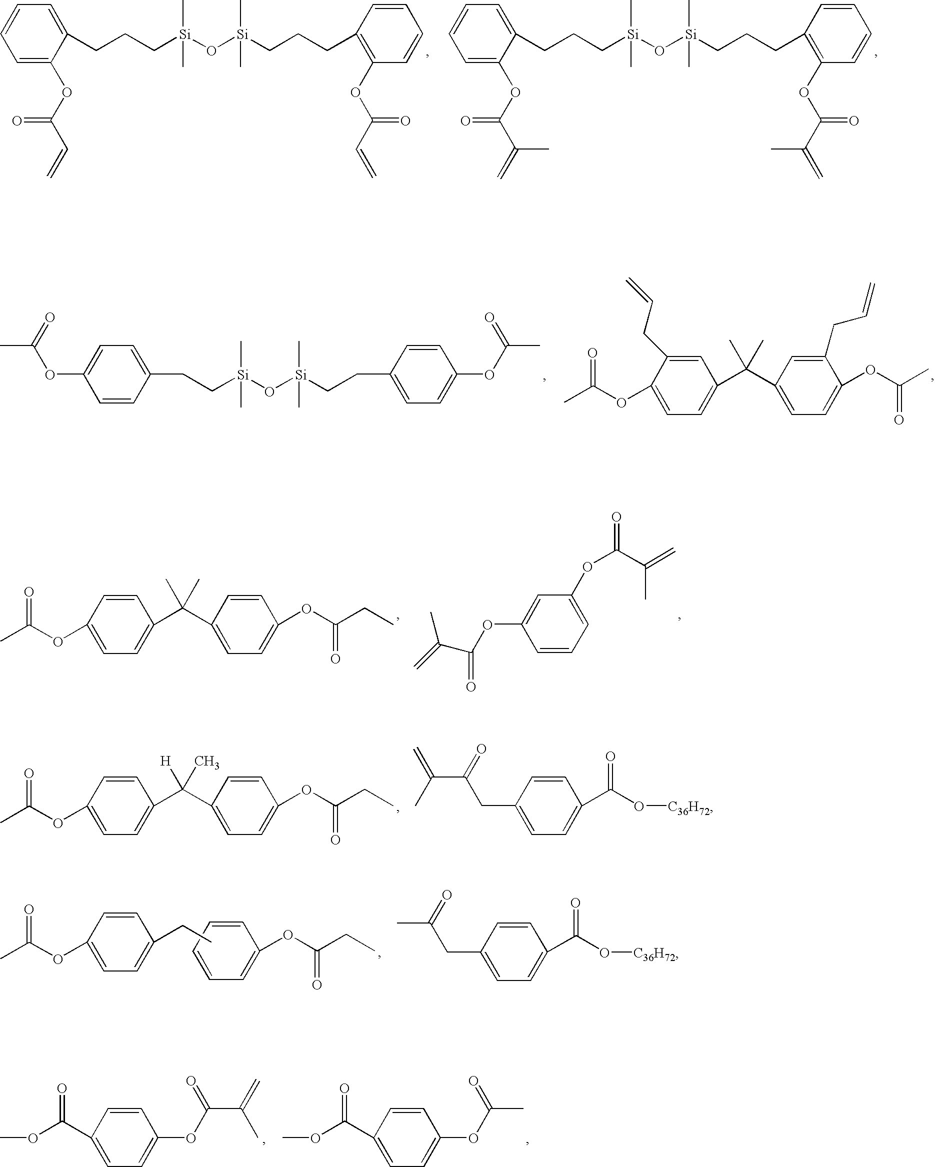

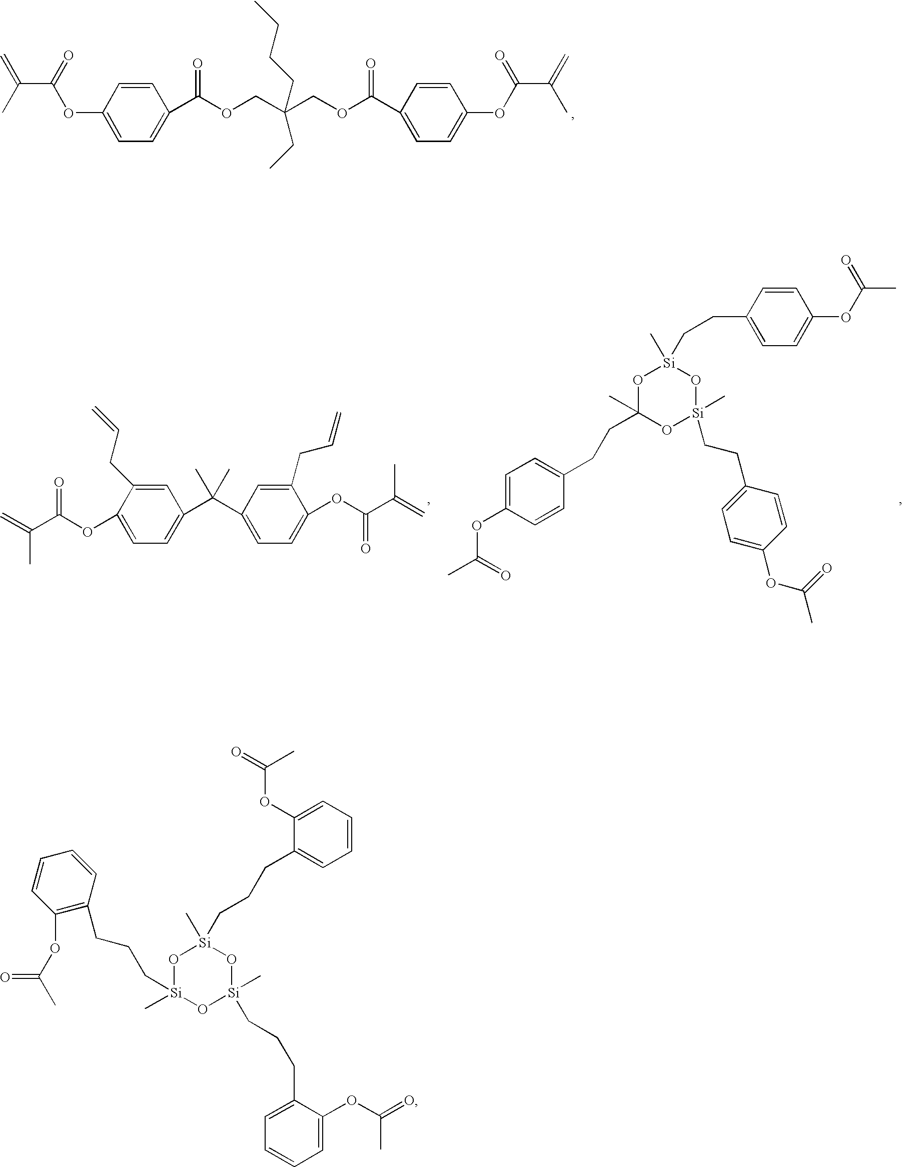

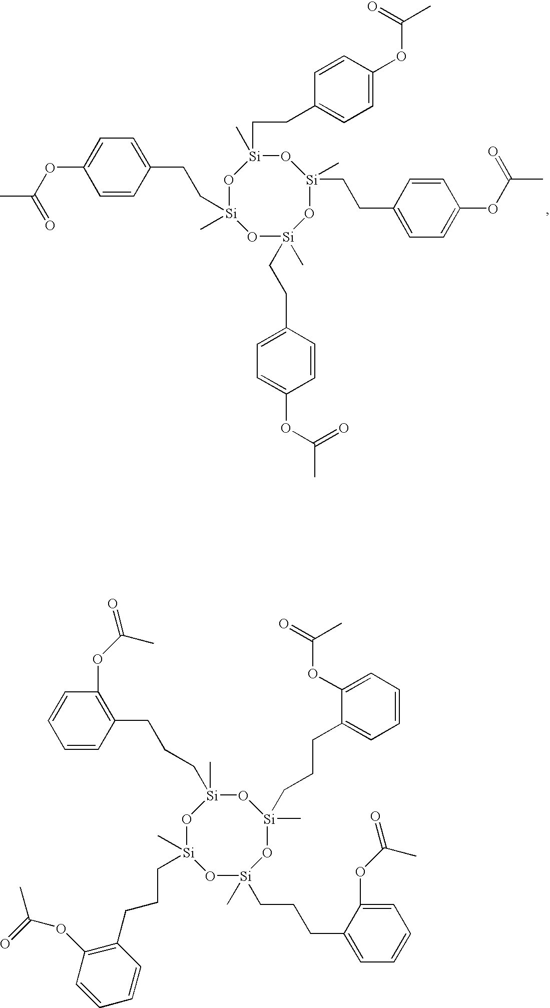

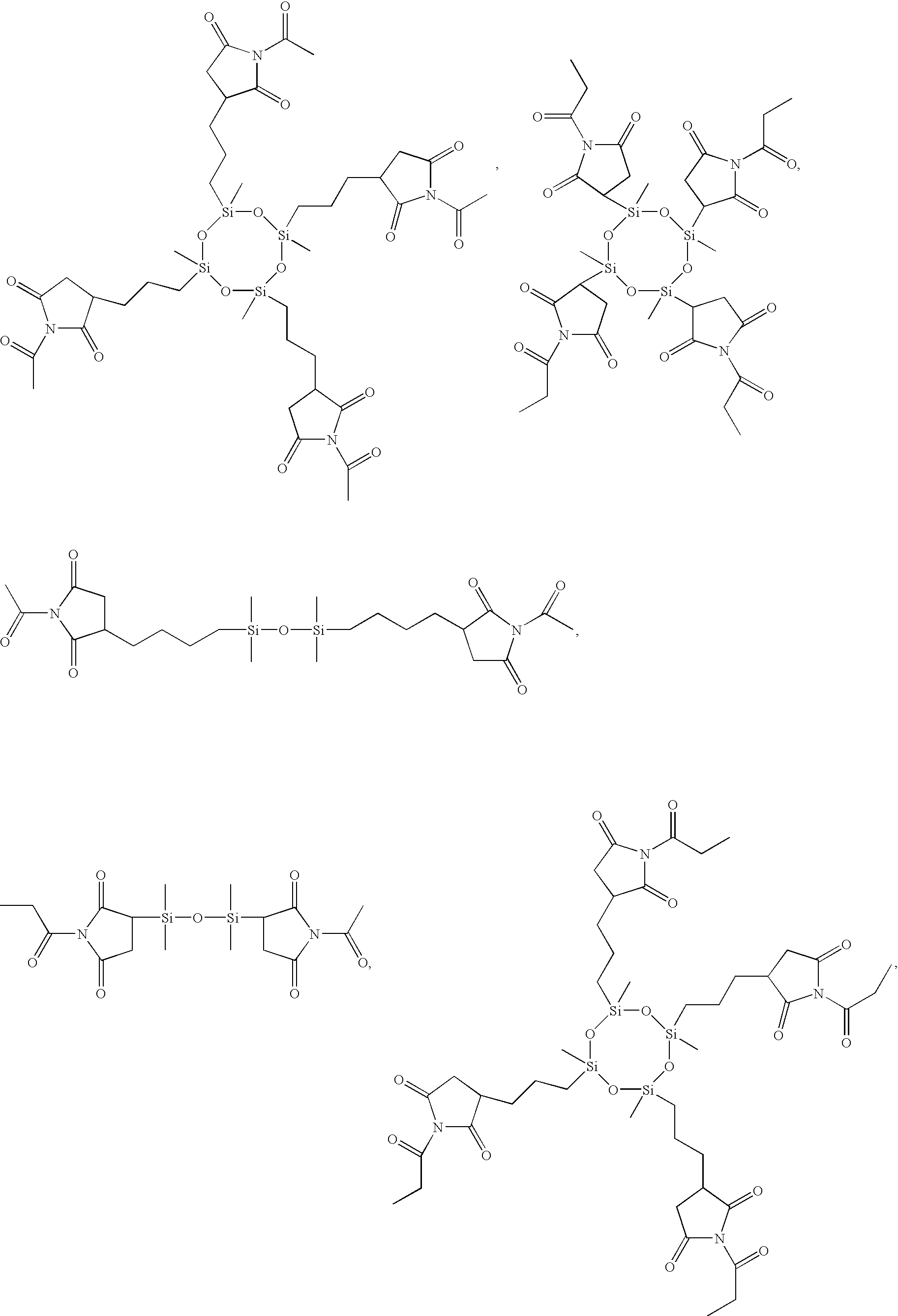

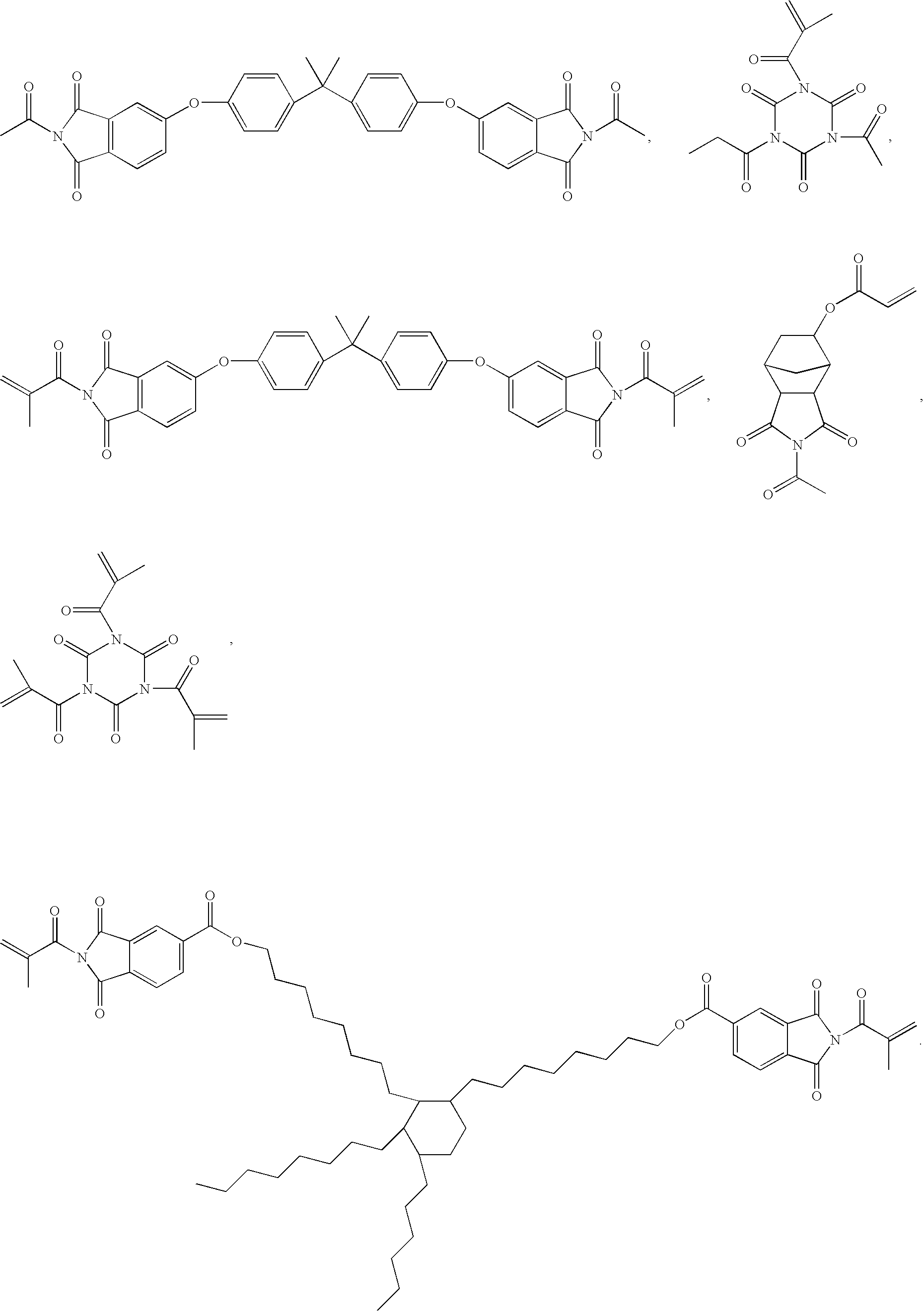

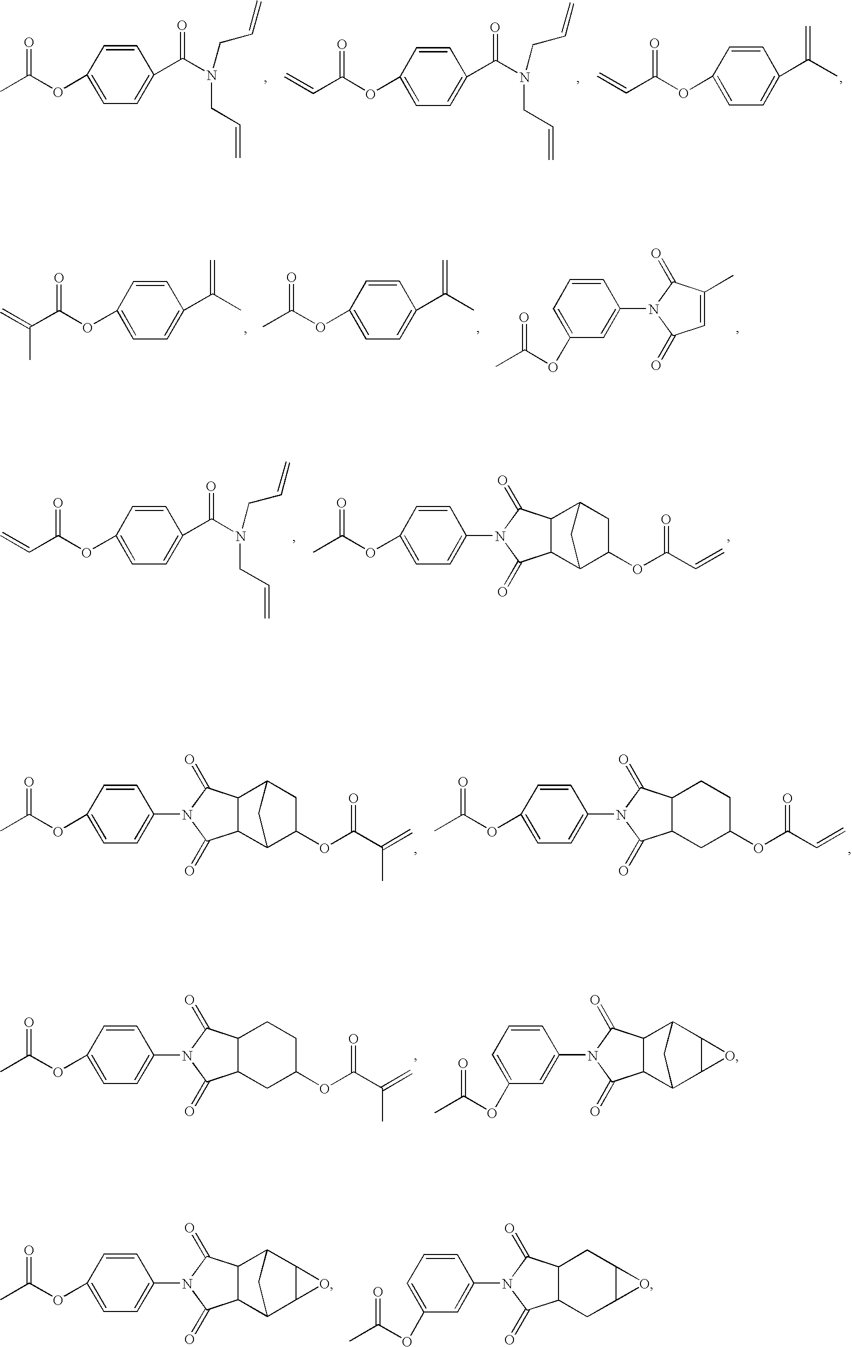

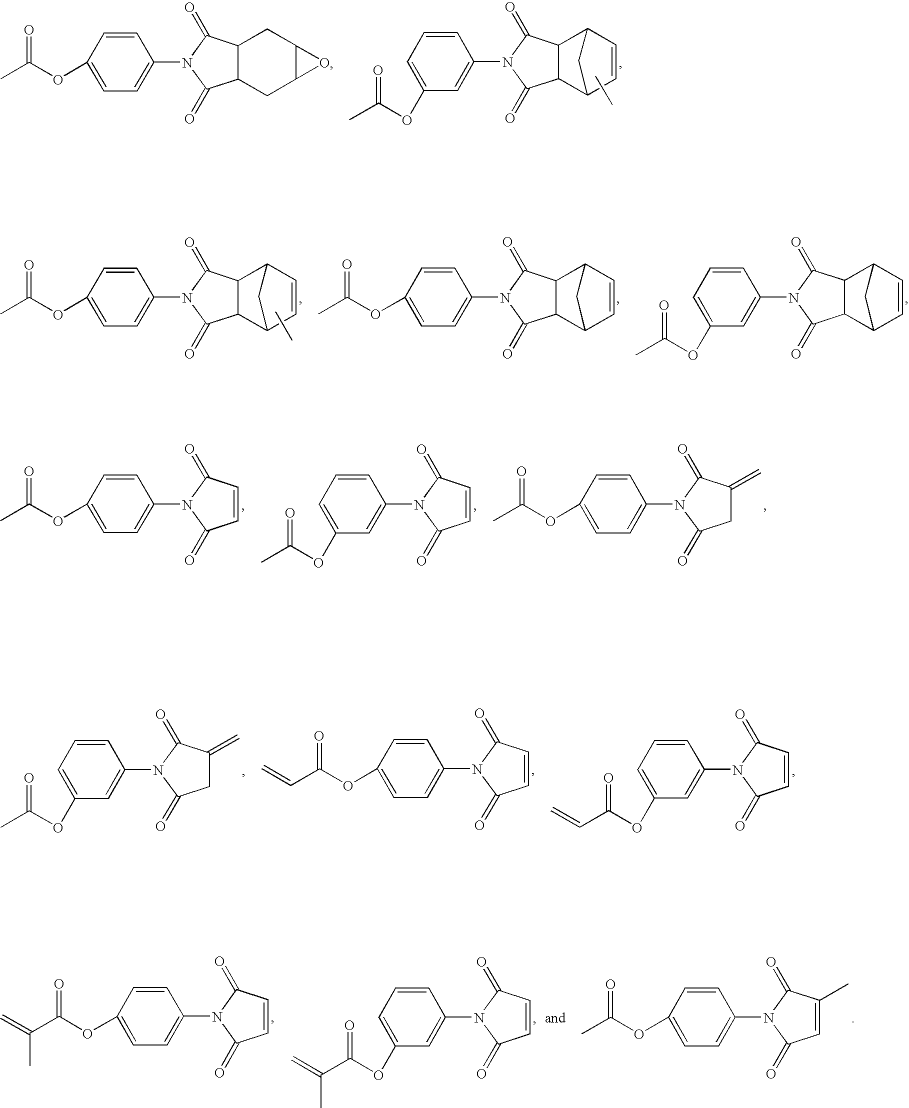

7. The curative of claim 1, selected from: 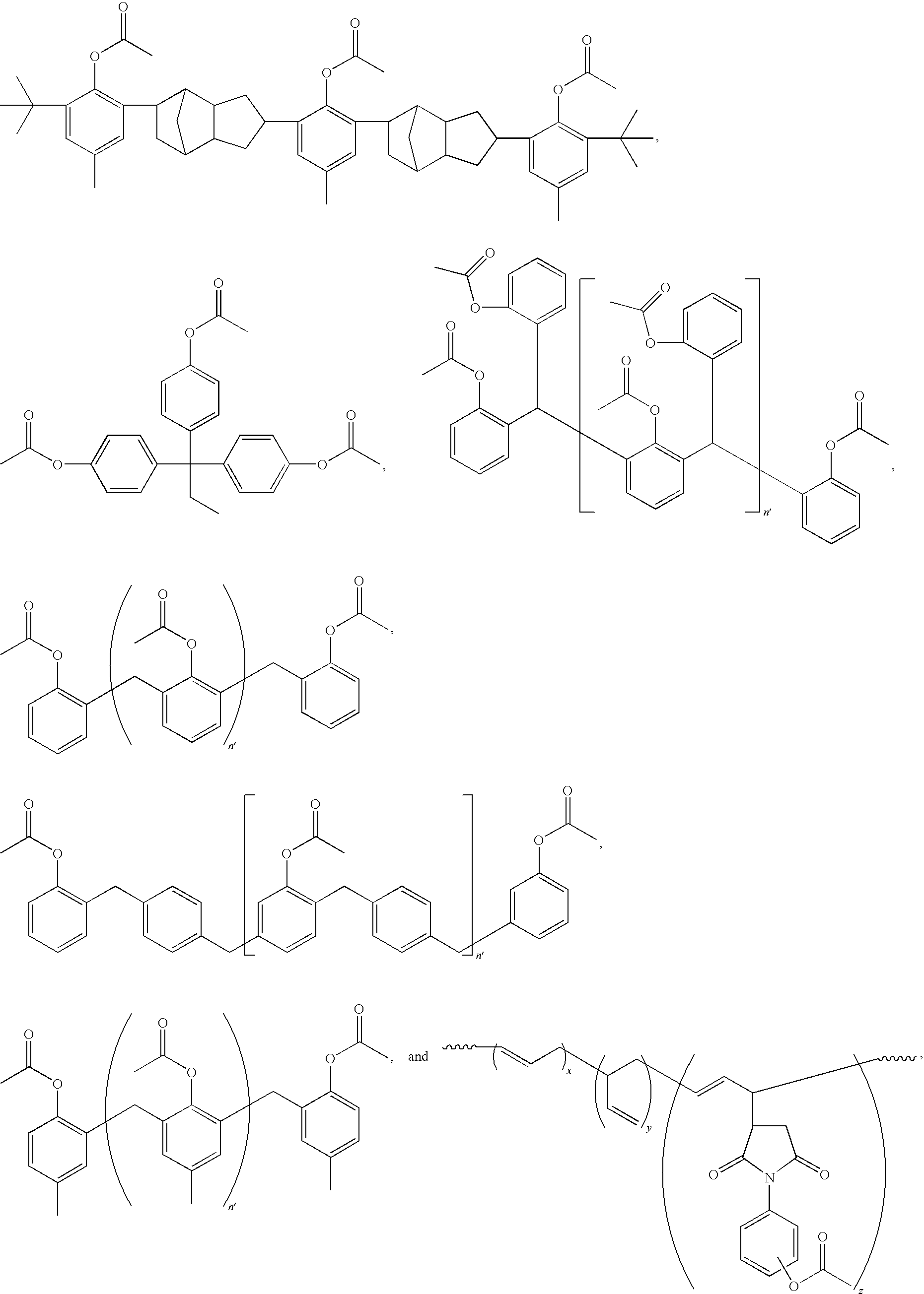
wherein:
n′ is 0 to about 10;
x is 4 to about 50;
y is 4 to about 50; and
z is 2 to about 40.

8. The curative of claim 1, selected from: 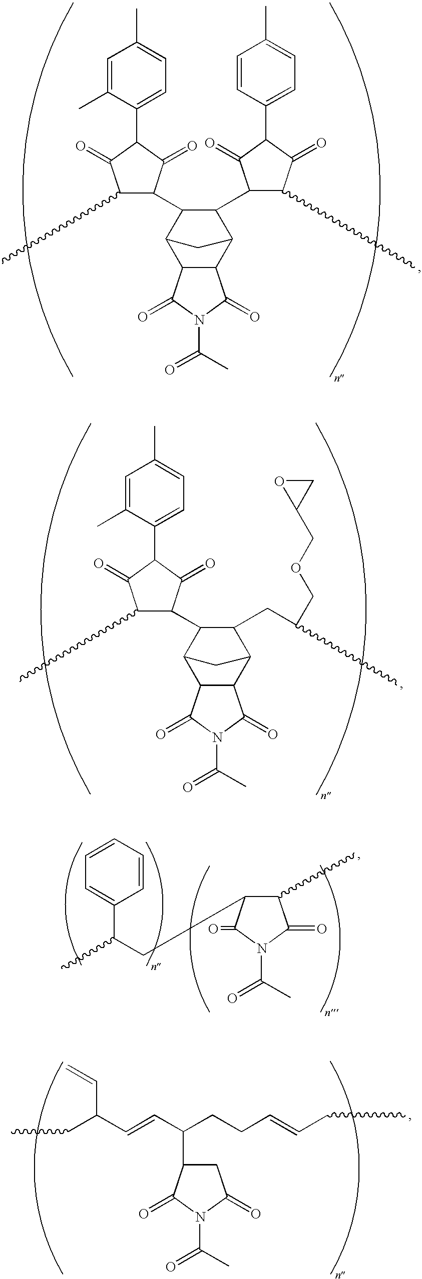 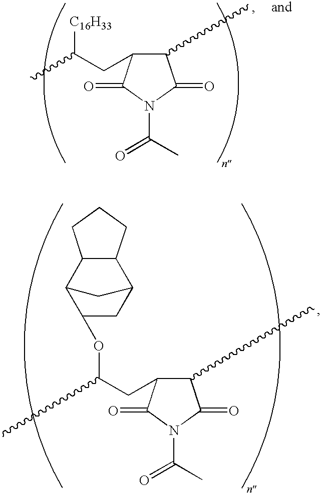
wherein each n″ and n′″ is independently 1 to about 10.

9. (canceled)

10. (canceled)

11. A composition comprising an epoxy resin or an oxetane resin and a curative of claim 1.

12. The composition of claim 11, wherein the epoxy comprises at least one of: a glycidyl ether epoxy, a cycloaliphatic epoxy, and an aliphatic epoxy.

13. (canceled)

14. (canceled)

15. (canceled)

16. (canceled)

17. The composition of claim 11, wherein the composition is an adhesive, a coating, a matrix resin or a composite resin.

18. The composition of claim 17, wherein the composition is selected from: an adhesive further comprising at least one compound selected from an acrylate, a methacrylate, a maleimide, a vinyl ether, a vinyl ester, a styrenic compound, an allyl functional compound, a phenol, an anhydride, a benzoxazine, and an oxazoline; a die attach paste adhesive further comprising a filler; an industrial or marine coating further comprising at least one of a filler, an extender and a pigment; and a composite resin further comprising at least one of carbon fiber, fiberglass or silica

19. (canceled)

20. The composition of claim 17, wherein the matrix resin is an industrial, marine, automotive, airline, aerospace, sporting goods, medical or dental matrix resin.

21. (canceled)

22. (canceled)

23. An assembly comprising a first article adhered to a second article by a cured aliquot of the composition of claim 11.

24. An article of manufacture coated with a cured layer of the composition of claim 11.

25. The article of manufacture of claim 24, wherein the article is a watercraft, automobile or airplane part.

26. An article of manufacture comprising a cured amount of the composition of claim 11.

27. The article of manufacture of claim 26, wherein the article is an industrial, marine, automotive, airline, aerospace, sporting goods, medical or dental article.

28. The article of manufacture of claim 26, further comprising at least one filler, extender, pigment, or reinforcing material.

29. (canceled)

30. (canceled)

31. (canceled)

32. (canceled)

33. A method for increasing the adhesion, decreasing the viscosity, reducing weight loss or decreasing the hydrophilicity of an epoxy resin or an oxetane resin, comprising combining a curative of claim 1 with the epoxy resin or the oxetane resin.

34. (canceled)

35. (canceled)

36. (canceled)
